(19) United States
(12) Patent Application Publication
(10) Pub. No.: US 2012/0270238 A1
(43) Pub. Date: Oct. 25, 2012

(54) Peptides That Bind the Alpha-Fetoprotein (AFP) Receptor and Uses Thereof

(76) Inventors: Ricardo J. Moro, Richmond (CA); Ralph H. Schmid, Vancouver (CA)

(21) Appl. No.: 13/502,306

(22) PCT Filed: Oct. 21, 2010
(86) PCT No.: PCT/IB2010/003152

§ 371 Date: Apr. 16, 2012

Related U.S. Application Data
(60) Provisional application No. 61/279,501, filed on Oct. 22, 2009.

(51) Int. Cl. C07K 7/06; G01N 33/574; C12N 11/02; C07K 1/14; C07K 9/00; C12N 7/00

(52) U.S. Cl. 435/7.23; 530/329; 530/322; 435/235.1; 435/177; 530/380

(57) ABSTRACT

The present invention provides an active binding sequence of mammalian alpha-fetoprotein (AFP) to the receptor of AFP (AFPr or RECAP). The sequence is embodied in peptides comprising Lys-Glx-Glx-Xaa-Leu-Ile-Asn (SEQ. ID. NO: 1) and variants thereof, wherein GIx means GIn or GIu, each GIx being selected independently of the other, and Xaa represents Phe or Leu. The peptides bind a site of the AFP receptor. This peptide can be used as a substitute for AFP in the detection, purification and imagining of RECAF. This peptide, as it binds to RECAF which is elevated in cancer cells, allows for a method of diagnostic determination of cancer or chemotherapeutic delivery using cytotoxic or radiological agents.

### FIELD OF THE INVENTION

The present invention relates to peptides that bind the alpha-fetoprotein (AFP) receptor, and uses thereof.

In particular, it relates to the detection, including targeting, of the alpha-fetoprotein (AFP) receptor in human and animal cells and to the purification and detection of the AFP receptor (AFPr) using a synthesized alpha-fetoprotein peptide sequence.

### BACKGROUND OF THE INVENTION

AFP is taken up by cells via a cell surface receptor (Villicampa, M. J., Moro, R., Naval, J., Failly-Cripin, Ch., Lampreave, F. and Uriel, J. Bioch. Biophys. Res Commun. 122, 1322 (1984)). The binding of AFP is known to be determined by a specific sequence in the amino acid chain, as it had been already shown that the carbohydrate moiety of AFP is not involved in the uptake of AFP into the cell.

The AFP receptor is known to be expressed by cancerous cells, and can migrate from the tumor site to body fluids, where it can be assayed to provide a detectable marker for the presence of cancer (WO-A-96/09551; and R. Moro et al., “Monoclonal antibodies against a widespread oncofetal antigen: the alpha-fetoprotein receptor”, Tumor Immunology, vol. 14, no. 2, 1 Jul. 1993, pages 116-130). The AFP receptor thus also provides a potential target for the targeted delivery of cytotoxic agents, for example cytotoxic drugs and radiological agents, to cancerous tumor cells in vivo.

Other peptides from AFP have been described, for example CCRDGVLDC (SEQ. ID. NO: 15) (WO-A-2004/03350, Dudich et al.), GIP peptide from amino acids 445-480 of AFP (U.S. Pat. No. 5,674,842 Mizejewski), and within GIP the antiestrotrophic fragment AA 472-479 (Mesfin et al., 2000 Biochim. Biophys. Acta, 1501: 33-34).

The synthetic peptide EMTXVNXGQ (SEQ. ID. NO: 16), where X is hydroxyl proline, has been described in US-A-2005/0036947.

Recombinantly-produced AFP peptides are covered in U.S. Pat. No. 6,534,479 to Murgita.

It is an aim of the present invention to provide useful new peptides which specifically bind the AFP receptor, and uses thereof.

### BRIEF DESCRIPTION OF THE INVENTION

The present invention provides in one aspect peptides comprising the sequence Lys-Glx-Glx-Xaa-Leu-Ile-Asn (SEQ. ID. NO: 1), wherein Glx means Gln or Glu, each Glx being selected independently of the other, and Xaa represents Phe or Leu, and variants thereof, that bind, preferably specifically, to the AFP receptor, preferably the human AFP receptor.

Peptides, including variants, according to the first aspect of the invention may be water-soluble or water-insoluble. The solubility can be selected according to the requirements of the use.

The peptides according to the present invention typically have a length less than about 25 amino acids, for example less than about 12 amino acids, more preferably less than about 10 amino acids.

In a second aspect the present invention provides an antibody capable of binding specifically to the peptide according to the first aspect of the invention.

In a third aspect the present invention provides an anti-idiotypic antibody raised against the antibody according to the second aspect of the invention and capable of binding specifically to the human AFP receptor.

In a fourth aspect the present invention provides a method for purifying AFPr which comprises binding said AFPr to the material (peptide, including antibody) according to the first or third aspect of the invention. The peptide/AFPr complex that results from this binding interaction can then be separated from the mixture. The AFPr can then be obtained from the complex in relatively pure form.

In a fifth aspect the present invention provides a method for detecting AFPr in which the material (peptide) according to the first or third aspect of the invention is first reacted with material containing AFPr to form a peptide/AFPr complex. The complex is then separated from the mixture.

By labeling the peptide, the peptide/AFPr complex can be detected. Alternatively, the binding can take place in the presence of, and in competition with, labeled AFPr, and the presence of AFPr in the sample can be detected by determining the relative binding of labeled and unlabelled AFPr. In both cases, the detection can be quantitative.

In a sixth aspect the present invention provides a method for detecting whether a biological sample obtained from a human or animal subject contains AFPr. In this aspect the sample and labeled AFPr is contacted with one or more specific binding partner for AFPr selected from anti-AFPr antibodies, anti-idiotypic antibodies with binding specificity for AFPr, AFP and fragments thereof with binding specificity for AFPr, and the material (peptide) according to the first aspect of the invention, with the proviso that at least one material (peptide) according to the first or third aspect of the present invention must be present, and the presence or absence of AFPr in the sample is detected by analyzing the competition for binding with the one or more specific binding partner, as between the sample and the labeled AFPr. At least one of the said specific binding partners may be immobilized on a solid support.

The method for detecting whether a biological sample contains AFPr can be used to detect pregnancy in a female human or animal. Alternatively, the method for detecting whether a biological sample contains AFPr can be used to detect, diagnose and treat cancer or other disease in a human or animal. To detect cancer, the possibility of the subject being pregnant would be eliminated by other tests or enquiries, and vice versa.

### DETAILED DESCRIPTION OF THE INVENTION

### AFP Receptor

The term “alpha-fetoprotein receptor” or “AFP receptor” (AFPr) used herein includes any synthetic or natural molecule, or portion of such molecule, that in its normal conformation or natural state shows specific binding to: (a) natural or synthetic alpha-fetoprotein (“AFP”); (b) a fragment of AFP; (c) a modification of AFP; (d) a modification of a fragment of AFP; (e) native or synthetic AFP bound to fatty acids or other molecules; or (f) a fragment of AFP bound to other fatty acids or other molecules.

The term “modification” used herein in relation to AFP include variants that maintain corresponding functionality but differ in their amino acid sequence from the wild-type or naturally occurring AFP molecule by insertion, substitution and/or deletion of amino acids that leave at least 80%, for example at least 90%, of the wild-type sequence unchanged, even if interrupted in places by the site(s) of said insertion, substitution and/or deletion.

### Specific Binding

“Specific binding” as used herein means that the molecules in question bind to each other in preference to, but not necessarily to the exclusion of, other molecules. The term includes any interaction between two molecules that: (i) becomes saturated as the concentration of one of the molecules is increased with respect to the other; and (ii) can be competed with the other molecule or an excess of the same molecule unlabeled.

### Antibody

The term “antibody” used herein includes antibody fragments such as Fab, F(ab)₂ or Fv.

Antibodies used in the present invention may be monoclonal or polyclonal. Chimeric and humanised forms of antibodies may be used if desired.

### Amino Acid Residue

The term “amino acid” or “amino acid residue” includes an amino acid residue contained in the group: alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues.

Synthetic amino acids are also encompassed by the term “amino acids” used herein.

### Peptides

The subject of the first and third aspects of the invention is a peptide or class of peptides that can be used in place of mammalian AFP for use in binding with the AFP receptor (AFPr or RECAF) for diagnostics, treatment or purification.

The term “peptide” used herein includes polypeptides and conjugated peptides in which the peptide moiety as defined in accordance with the present invention is conjugated to a non-peptide moiety, as described in more detail below.

The present invention provides in one aspect a peptide comprising the sequence Lys-Glx-Glx-Xaa-Leu-Ile-Asn (SEQ. ID. NO: 1) wherein Glx means Gln or Glu, each Glx being selected independently of the other, and Xaa represents Phe or Leu, and variants thereof, that binds, preferably specifically, to the human AFP receptor.

The term “variants” used herein in relation to the peptide of the present invention includes peptides that differ from SEQ. ID. NO: 1 but maintain corresponding functionality, by having at least one insertion, substitution and/or deletion of amino acids in the above heptapeptide motif of SEQ. ID. NO: 1, such that at least 4 contiguous amino acids of the heptapeptide motif are maintained in the same order as in SEQ. ID. NO: 1.

Each variant retains substantially the activity of binding to a mammalian AFP receptor and/or of detectably affecting the binding of AFP to AFPr. The peptides of the present invention may be produced by any suitable method known in the art, such as chemical synthesis and/or recombinant DNA technology.

A typical variant of a peptide differs in amino acid sequence from another polypeptide. Provided that the functionality mentioned in the previous paragraph is maintained, one or more amino acids of the said amino acid sequence thereof may be substituted by one or more other amino acids. Such amino acids may be selected from the naturally occurring amino acids, for example selected from the group T, M, H, A, G, V, C, K, Q, E, F, L, I, N and D. For example, such variants can, but need not to contain one or more conservative amino acid substitutions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a peptide is preferably replaced with another amino acid residue from the same side chain family. The peptide variants of this invention can be tested for binding activity to AFPr.

Examples of variants of SEQ ID. NO: 1 are peptides comprising heptapeptide motif Lys-Glx-Glx-Xaa-Ile-Asp-Leu (SEQ. ID. NO: 2), wherein Glx and Xaa are as defined above in relation to SEQ. ID. NO: 1.

The peptides can consist of or contain the sequence KQEFLIN (SEQ. ID. NO: 3).

The peptide may, for example, be any fragment from the 609 amino acid sequence shown in FIG. 2 (SEQ. ID. NO: 4), provided that the sequence KQEFLIN (SEQ. ID. NO: 3) (amino acids 549-555 of SEQ. ID. NO: 4) is conserved.

In one embodiment, the peptide may consist of or contain the sequence HKDLCQAQGVALQTMKQEFLIN (SEQ. ID. NO: 5) (amino acids 534-555 of SEQ. ID. NO: 4). This sequence is referred to as Fragment #3 in FIG. 4.

In one embodiment, the peptide may consist of or contain the sequence LQTMKQEFLIN (SEQ. ID. NO: 6) (amino acids 545-555 of SEQ. ID. NO: 4). This sequence is referred to as Fragment #4 in FIG. 4.

In one embodiment, the peptide may consist of or contain the sequence TMKQEFLIN (SEQ. ID. NO: 7) (amino acids 547-555 of SEQ. ID. NO: 4). This sequence is referred to as Fragment #10 in FIG. 4.

In one embodiment, the peptide may consist of or contain the sequence LQTMKQELLIN (SEQ. ID. NO: 8) (amino acids 545-555 of SEQ. ID. NO: 4 with aa552 substituted F→L). This sequence is referred to as Fragment #17 in FIG. 4.

In one embodiment, the peptide may consist of or contain the sequence KQELLIN (SEQ. ID. NO: 9) (amino acids 549-555 of SEQ. ID. NO: 4 with aa552 substituted F→L). This sequence is referred to as Fragment #16 in FIG. 4.

In one embodiment, the peptide may consist of or contain the sequence KEEFLIN (SEQ. ID. NO: 10).

In one embodiment, the peptide may consist of or contain the sequence KEQFLIN (SEQ. ID. NO: 11).

In one embodiment, the peptide may consist of or contain the sequence KQQFLIN (SEQ. ID. NO: 12).

In one embodiment, the peptide may consist of or contain the sequence KQQFIDL (SEQ. ID. NO: 13).

In one embodiment, the peptide may consist of or contain the sequence KQQLIDL (SEQ. ID. NO: 14).

At least some of the peptides according to the present invention (e.g. Fragment #4) are found to be soluble in aqueous media without the need for an organic solvent. This is a substantial and unexpected advantage when it comes to developing compositions for use in assays, diagnostic agents, therapeutic agents and the like.

The peptide according to the first aspect of the present invention may include one or more additional amino acids at the N-terminal of the heptapeptide motif or variant thereof, or one or more additional amino acids at the C-terminal of the heptapeptide motif or variant thereof, or one or more additional amino acids at both the N- and the C-terminals of the heptapeptide motif or variant thereof. The one or more amino acids may be selected from all natural and synthetic amino acids, and when more than one amino acid is present at either or both terminals they may be present in any sequence. When one or more additional amino acids are present at both terminals, they and their sequences are independently selected from each other so that the amino acid(s) and, if more than one amino acid, sequence at the N-terminal can be then same as or different from the amino acid(s) and, if more than one amino acid, sequence at the C-terminal. The addition of amino acids at one or both termini can be used to control the water-solubility of the peptide and the adsorption of the peptide onto solid phases. Appropriate selection of the additional amino acids and other moieties can make the peptide more or less water-soluble.

Functional groups can be incorporated into the peptides according to the present invention, for example functional groups which permit the peptide to be covalently linked to a surface or to other molecules or species via one or both ends of the peptide.

The peptide according the first aspect of the present invention may include one or more other moieties to provide specific functionality. Normally, any such one or more other moieties that may be present will not interfere with the functionality of the peptide to bind to a mammalian AFPr or to detectably compete with AFP for binding to mammalian AFPr. For example, the peptide may include one or more Cys (C) amino acid in any peptide portion, to enable disulfide cross-linking between portions or molecules. In another example, the peptide may include one or more Tyr (T), to enable radiolabeling of the peptide to allow its detection. In one embodiment, one or more radiolabelled tyrosine (Y) may be provided in the peptide according to the first aspect of the present invention. It is preferred that such other moieties will be present in portions of the peptide other than the heptapeptide motif Lys-Glx-Glx-Xaa-Leu-Ile-Asn (SEQ. ID. NO: 1) or any variant thereof having at least one insertion, substitution and/or deletion of amino acids therein such that at least 4 contiguous amino acids of the heptapeptide motif are maintained in the same order as in SEQ. ID. NO: 1, for example the heptapeptide motif Lys-Glx-Glx-Xaa-Ile-Asp-Leu (SEQ. ID. NO: 2).

The peptide of the present invention is capable of binding to the alpha-fetoprotein (AFP) receptor (AFPr) as defined above and/or of detectably affecting the binding of AFP to AFPr, for example detectably competing with AFP for binding to AFPr.

### Peptide Synthesis

The peptides of the present invention may be produced by any suitable method known in the art, such as chemical synthesis and/or recombinant DNA technology. For example, the inventive peptide can be synthesized using solid phase peptide synthesis techniques (e.g., Fmoc). Alternatively, the peptide can be synthesized using recombinant DNA technology (e.g., using bacterial or eukaryotic expression systems).

A nucleotide sequence encoding a polypeptide of the invention may be constructed by isolating or synthesizing a nucleotide sequence encoding the parent peptide and then changing the nucleotide sequence so as to effect introduction (i.e. insertion or substitution) or removal (i.e. deletion or substitution) of the relevant amino acid residue(s).

Methods for solid state protein synthesis and recombinant protein synthesis are well-known in the art. For example, “Molecular Cloning, A Laboratory Manual” (Sambrook et al., 3d Edition, Cold Spring Harmor Press), is a well-known reference detailing many suitable techniques for recombinant production of polypeptides. A variant of a peptide may be naturally occurring or it may be a variant that is not known to occur naturally. Non-naturally occurring variants of peptides may be made by direct synthesis, or alternatively, mutations can be introduced randomly along all or part of a peptide of this invention, such as by saturation mutagenesis or site-directed mutagenesis in accordance with conventional methods.

Independent of the method of production, the resultant variants can be screened for the ability of binding to AFPr to identify variants of this invention.

To prepare a recombinant peptide, one can clone a nucleic acid encoding the peptide in an expression vector, in which the nucleic acid is operably linked to a regulatory sequence suitable for expressing the polypeptide in a host cell. One can then introduce the vector into a suitable host cell to express the peptide. Alternatively, the nucleic acid can be linked to another nucleic acid encoding a fusion partner, e.g., glutathione-S-transferase (GST), T7 tag, 6.times.-His epitope tag, M13 Gene 3 protein, or an immunoglobulin heavy chain constant region. The resultant fusion nucleic acid expresses in suitable host cells a fusion protein. Suitable host cells are those that are resistant to this apoptotic peptide and can be obtained using screening methods known in the art. The expressed recombinant peptides can be purified from the host cell by methods such as ammonium sulfate precipitation and fractionation column chromatography. See Goeddel, (1990) Gene Expression Technology: Methods in Enzymology 185, Academic Press, San Diego, Calif. Water-soluble polypeptides are then prepared by the method described in U.S. application Ser. No. 10/449,531 and Wang et al., 2003, Vaccine 21, 3721-3729t. An isolated fusion protein can be further treated, e.g., by enzymatic digestion, to remove the fusion partner and obtain the recombinant polypeptide of this invention.

In one embodiment of this aspect of the present invention, the peptide may be an isolated peptide. In particular, the isolated peptide may be a peptide molecule that is in a non-natural environment. The non-natural environment may, for example, be a synthetic peptide molecule, a recombinantly expressed peptide molecule, a cell culture, a recombinant cell or organism, a pharmaceutical composition, a foreign host cell or organism. The isolated peptide may optionally, but not essentially, be in a purified or partially purified condition, for example the predominant chemical constituent of a mixture.

In one embodiment of this aspect of the present invention, a peptide can be branched, or cyclic, with or without branching. Cyclic, branched and non-branched polypeptides can result from post-translational natural processes and can be made by entirely synthetic methods as well. The peptide can be made as a polymer via branched lysine(s) through F-moc chemistry. The peptide backbone of a molecule of the invention can be constructed using L-amino acids or D-amino acids or peptide-like mimetics in order to resist degradation.

However it is made, the inventive peptide can be isolated and/or purified (or substantially isolated and/or substantially purified). Accordingly, the invention provides the peptide of the first aspect of the present invention in isolated or substantially isolated form (e.g., substantially isolated from other peptides or impurities). The peptide can be isolated from other peptides as a result of solid phase protein synthesis, for example. Alternatively, the peptide can be substantially isolated from other proteins after cell lysis from recombinant production.

Standard methods of protein purification (e.g., HPLC) can be employed to substantially purify the inventive peptides. Thus, a preparation of the peptide according to the present invention preferably is at least 90% (by weight) free of other peptides and/or contaminants, and more preferably is at least about 95% (by weight) free of other peptides and/or contaminants (such as at least about 97% or 98% (by weight) free of other peptides and/or contaminants). A preparation of the peptide according to the present invention may suitably be greater than 90% (by weight) pure.

### Peptides Conjugated to a Non-Peptide Moiety

In one embodiment of this aspect of the invention, a peptide of this invention can be conjugated to a non-peptide moiety. Such a variant is encompassed within the term “peptide” used herein.

The peptide can be conjugated to a non-peptide moiety using N-hydroxysuccinimide ester (NHS) or other nucleophiles that will form a covalent linkage with the N-terminal of the peptide. For example, Fragment #4 (see FIG. 4) has been conjugated to acridinium and biotin using NHS and to horseradish peroxidise (HRP) using standard periodate treatment.

In preferred embodiments, the non-peptide moiety to which the peptide according to the present invention may be conjugated to a polymer molecule, a lipophilic compound, a sugar moiety (e.g. preparable by way of in vivo glycosylation) and an organic derivatizing agent. All of these agents may confer desirable properties to the peptide, in particular increased binding to AFPr, increased functional in vivo half-life and/or increased plasma half-life. The peptide is normally conjugated to only one type of non-peptide moiety, but may also be conjugated to two or more different types of non-peptide moieties, e.g. to a polymer molecule and a sugar moiety, to a lipophilic group and a sugar moiety, to an organic derivatizing agent and a sugar moiety, to a lipophilic group and a polymer molecule, etc. The process of conjugation or a peptide according to the present invention to two or more different non-peptide moieties may be done simultaneously or sequentially.

A peptide according to the invention which is conjugated to a non-peptide moiety may be produced in vivo by culturing an appropriate host cell under conditions conducive for the expression of the peptide, and recovering the conjugated peptide. Such a manufacturing process may be appropriate where the conjugated peptide comprises at least one N- or O-glycosylation site and the host cell is a eukaryotic host cell capable of in vivo glycosylation. Alternatively or additionally a conjugated peptide can be subjected to conjugation to a non-peptide moiety in vitro.

### Polymers

A polymer molecule to be coupled to the peptide may be any suitable polymer molecule, such as a natural or synthetic homo-polymer or hetero-polymer, typically with a molecular weight in the range of about 300-100,000 Da, such as about 500-20,000 Da, more preferably in the range of about 500-15,000 Da, even more preferably in the range of about 2-12 kDa, such as in the range of about 3-10 kDa When the term “about” is used herein in connection with a certain molecular weight, the word “about” indicates an approximate average molecular weight and reflects the fact that there will normally be a certain molecular weight distribution in a given polymer preparation.

Examples of homo-polymers include a polyol (i.e. poly-OH), a polyamine (i.e. poly-NH₂) and a polycarboxylic acid (i.e. poly-COOH). A hetero-polymer is a polymer comprising different coupling groups, such as a hydroxyl group and an amine group.

Examples of suitable polymer molecules include polymer molecules selected from the group consisting of polyalkylene oxide (PAO), including polyalkylene glycol (PAG), such as polyethylene glycol (PEG) and polypropylene glycol (PPG), branched PEGs, poly-vinyl alcohol (PVA), poly-carboxylate, poly-(vinylpyrolidone), polyethylene-co-maleic acid anhydride, polystyrene-co-maleic acid anhydride, dextran, including carboxymethyl-dextran, or any other biopolymer suitable for reducing immunogenicity and/or increasing functional in vivo half-life and/or serum half-life. Another example of a polymer molecule is human albumin or another abundant plasma protein. Generally, polyalkylene glycol-derived polymers are biocompatible, non-toxic, non-antigenic, non-immunogenic, have various water solubility properties, and are easily excreted from living organisms.

PEG is a preferred polymer molecule, since it has only few reactive groups capable of cross-linking compared to, e.g., polysaccharides such as dextran. In particular, mono-functional PEG, e.g. methoxypolyethylene glycol (mPEG), is of interest since its coupling chemistry is relatively simple (only one reactive group is available for conjugating with attachment groups on the peptide). Consequently, as the risk of cross-linking is eliminated, the resulting conjugated variants are more homogeneous and the reaction of the polymer molecules with the conjugated peptide is easier to control.

To effect covalent attachment of the polymer molecule(s) to the peptide according to the present invention, the hydroxyl end groups of the polymer molecule may be provided in activated form, i.e. with reactive functional groups (examples of which include primary amino groups, hydrazide (HZ), thiol, succinate (SUC), succinimidyl succinate (SS), succinimidyl succinamide (SSA), succinimidyl propionate (SPA), succinimidyl butyrate (SBA), succinimidy carboxymethylate (SCM), benzotriazole carbonate (BTC), N-hydroxysuccinimide (NHS), aldehyde, nitrophenylcarbonate (NPC), and tresylate (TRES)). Suitable activated polymer molecules are commercially available, e.g. from Shearwater Polymers, Inc., Huntsville, Ala., USA, or from PoIyMASC Pharmaceuticals plc, UK.

Alternatively, the polymer molecules can be activated by conventional methods known in the art, e.g. as disclosed in WO-A-90/13540. Specific examples of activated linear or branched polymer molecules for use in the present invention are described in the Shearwater Polymers, Inc. 1997 and 2000 Catalogs (Functionalized Biocompatible Polymers for Research and pharmaceuticals, Polyethylene Glycol and Derivatives, incorporated herein by reference).

Specific examples of activated PEG polymers include the following linear PEGs: NHS-PEG (e.g. SPA-PEG, SSPA-PEG, SBA-PEG, SS-PEG, SSA-PEG, SC-PEG, SG-PEG, and SCM-PEG), and NOR-PEG, BTC-PEG, EPDX-PEG, NCO-PEG, NPC-PEG, CDI-PEG, ALD-PEG, TRES-PEG, VS-PEG, IODO-PEG, and MAL-PEG, and branched PEGs such as PEG2-NHS and those disclosed in U.S. Pat. Nos. 5,932,462 and 5,643,575, both of which are incorporated herein by reference. Furthermore, the following publications, incorporated herein by reference, disclose useful polymer molecules and/or PEGylation chemistries: U.S. Pat. No. 5,824,778, U.S. Pat. No. 5,476,653, WO-A-97/32607, EP-A-229,108, EP-A-402,378, U.S. Pat. Nos. 4,902,502, 5,281,698, 5,122,614 and 5,219,564, WO-A-92/16555, WO-A-94/04193, WO-A-94/14758, WO-A-94/17039, WO-A-94/18247, WO-A-94/28024, WO-A-95/00162, WO-A-95/11924, WO-A-95/13090, WO-A-95/33490, WO-A-96/00080, WO-A-97/18832, WO-A-98/41562, WO-A-98/48837, WO-A-99/32134, WO-A-99/32139, WO-A-99/32140, WO-A-96/40791, WO-A-98/32466, WO-A-95/06058, EP-A-439508, WO-A-97/03106, WO-A-96/21469, WO-A-95/13312, EP-A-921131, U.S. Pat. No. 5,736,625, WO-A-98/05363, EP-A-809996, U.S. Pat. No. 5,629,384, WO-A-96/41813, WO-A-96/07670, U.S. Pat. Nos. 5,473,034 and 5,516,673, EP-A-605963, U.S. Pat. No. 5,382,657, EP-A-510356, EP-A-400472, EP-A-183503 and EP-A-154316.

Specific examples of activated PEG polymers particularly preferred for coupling to cysteine residues, include the following linear PEGs: vinylsulfone-PEG (VS-PEG), preferably vinylsulfone-mPEG (VS-mPEG); maleimide-PEG (MAL-PEG), preferably maleimide-mPEG (MAL-mPEG) and orthopyridyl-disulfide-PEG (OPSS-PEG), preferably orthopyridyl-disulfide-mPEG (OPSS-mPEG). Typically, such PEG or mPEG polymers will have a size of about 5 kDa, about 10 kD, about 12 kDa or about 20 kDa.

The conjugation of a peptide according to the present invention to an activated polymer is conducted by use of any conventional method, e.g. as described in the following references (which also describe suitable methods for activation of polymer molecules): Harris and Zalipsky, eds., Poly(ethylene glycol) Chemistry and Biological Applications, AZC, Washington; R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y.).

The skilled person will be aware that the activation method and/or conjugation chemistry to be used depends on the attachment group(s) of the variant polypeptide (examples of which are given further above), as well as the functional groups of the polymer (e.g. being amine, hydroxyl, carboxyl, aldehyde, sulfhydryl, succinimidyl, maleimide, vinysulfone or haloacetate). The PEGylation may be directed towards conjugation to all available attachment groups on the variant polypeptide (i.e. such attachment groups that are exposed at the surface of the polypeptide) or may be directed towards one or more specific attachment groups, e.g. the N-terminal amino group as described in U.S. Pat. No. 5,985,265 or to cysteine residues. Furthermore, the conjugation may be achieved in one step or in a stepwise manner (e.g. as described in WO-A-99/55377).

For PEGylation to cysteine residues (see above) the FVII or FVIIa variant is usually treated with a reducing agent, such as dithiothreitol (DDT) prior to PEGylation. The reducing agent is subsequently removed by any conventional method, such as by desalting. Conjugation of PEG to a cysteine residue typically takes place in a suitable buffer at pH 6-9 at temperatures varying from 4° C. to 25° C. for periods up to 16 hours.

The conjugation can readily be designed to produce the desired molecule with respect to the number of non-peptide moieties attached, the size and form of such molecules (e.g. whether they are linear or branched), and the attachment site(s) in the peptide. The molecular weight of the non-peptide moiety to be used may, e.g., be chosen on the basis of the desired effect to be achieved. For instance, if the primary purpose of the conjugation is to achieve a conjugated variant having a high molecular weight (e.g. to reduce renal clearance) it is usually desirable to conjugate as few high molecular weight non-peptide moieties as possible to obtain the desired molecular weight. When a high degree of shielding is desirable this may be obtained by use of a sufficiently high number of low molecular weight non-peptide moieties (e.g. with a molecular weight of from about 300 Da to about 5 kDa, such as a molecular weight of from 300 Da to 2 kDa).

In connection with conjugation to only a single attachment group on the protein (e.g. the N-terminal amino group), it may be advantageous that the polymer molecule, which may be linear or branched, has a high molecular weight, preferably about 10-25 kDa, such as about 15-25 kDa, e.g. about 20 kDa.

Normally, the polymer conjugation is performed under conditions aimed at reacting as many of the available polymer attachment groups with polymer molecules. This is achieved by means of a suitable molar excess of the polymer relative to the peptide. Typically, the molar ratios of activated polymer molecules to peptide are up to about 1000:1, such as up to about 200:1, or up to about 100:1. In some cases the molar ratio may be somewhat lower, however, such as up to about 50:1, 10:1, 5:1, 2:1 or 1:1.

It is also contemplated according to the invention to couple the polymer molecules to the polypeptide through a linker. Suitable linkers are well known to the skilled person. A preferred example is cyanuric chloride (Abuchowski et al., (1977), J. Biol. Chem., 252, 3578-3581; U.S. Pat. No. 4,179,337; Shafer et al., (1986), J. Polym. Sci. Polym. Chem. Ed., 24, 375-378).

Subsequent to the conjugation, residual activated polymer molecules can be blocked according to methods known in the art, e.g. by addition of primary amine to the reaction mixture, and the resulting inactivated polymer molecules are removed by a suitable method.

It will be understood that depending on the circumstances, e.g. the amino acid sequence of the peptide according to the present invention, the nature of the activated PEG compound being used and the specific PEGylation conditions, including the molar ratio of PEG to peptide, varying degrees of PEGylation may be obtained, with a higher degree of PEGylation generally being obtained with a higher ratio of PEG to peptide. The PEGylated peptides resulting from any given PEGylation process will, however, normally comprise a stochastic distribution of conjugated peptides having slightly different degrees of PEGylation.

### Sugar Moieties

In order to achieve in vivo glycosylation of a peptide of this invention comprising one or more glycosylation sites the nucleotide sequence encoding the peptide must be inserted in a glycosylating, eucaryotic expression host. The expression host cell may be selected from fungal (filamentous fungal or yeast), insect or animal cells or from transgenic plant cells. In one embodiment the host cell is a mammalian cell, such as a CHO cell, BHK or HEK, e.g. HEK 293, cell, or an insect cell, such as an SF9 cell, or a yeast cell, e.g. S. cerevisiae or Pichia pastoris, or any of the host cells mentioned hereinafter.

Covalent in vitro coupling of sugar moieties (such as dextran) to amino acid residues of the variant polypeptide may also be used, e.g. as described, for example in WO-A-87/05330 and in Aplin et al., CRC Crit. Rev. Biochem, pp. 259-306, 1981. The in vitro coupling of sugar moieties or PEG to protein- and peptide-bound Gln-residues can be carried out by transglutaminases (TGases). Transglutaminases catalyse the transfer of donor amine-groups to protein- and peptide-bound Gln-residues in a so-called cross-linking reaction. The donor-amine groups can be protein- or peptide-bound, such as the .epsilon.-amino-group in Lys-residues or it can be part of a small or large organic molecule. An example of a small organic molecule functioning as amino-donor in TGase-catalysed cross-linking is putrescine (1,4-diaminobutane). An example of a larger organic molecule functioning as amino-donor in TGase-catalysed cross-linking is an amine-containing PEG (Sato et al., 1996, Biochemistry 35, 13072-13080).

TGases, in general, are highly specific enzymes, and not every Gln-residues exposed on the surface of a protein is accessible to TGase-catalysed cross-linking to amino-containing substances. On the contrary, only few Gin-residues are naturally functioning as TGase substrates but the exact parameters governing which Gln-residues are good TGase substrates remain unknown. Thus, in order to render a protein susceptible to TGase-catalysed cross-linking reactions it is often a prerequisite at convenient positions to add stretches of amino acid sequence known to function very well as TGase substrates. Several amino acid sequences are known to be or to contain excellent natural TGase substrates e.g. substance P, elafin, fibrinogen, fibronectin, α₂-plasmin inhibitor, α-caseins, and α-caseins.

### Organic Derivatizing Agents

Covalent modification of the peptide according to the present invention may be performed by reacting one or more attachment groups of the peptide with an organic derivatizing agent. Suitable derivatizing agents and methods are well known in the art. For example, cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(4-imidazoyl)-propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole. Histidyl residues are derivatized by reaction with diethylpyrocarbonate at pH 5.5-7.0 because this agent is relatively specific for the histidyl side chain. Para-bromophenacyl bromide also is useful. The reaction is preferably performed in 0.1 M sodium cacodylate at pH 6.0. Lysinyl and amino terminal residues are reacted with succinic or other carboxylic acid anhydrides. Derivatization with these agents has the effect of reversing the charge of the lysinyl residues. Other suitable reagents for derivatizing α-amino-containing residues include imidoesters such as methyl picolinimidate, pyridoxal phosphate, pyridoxal, chloroborohydride, trinitrobenzenesulfonic acid, O-methylisourea, 2,4-pentanedione and transaminase-catalyzed reaction with glyoxylate. Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group.

Furthermore, these reagents may react with the groups of lysine as well as the arginine guanidino group. Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylphenyl)carbodiimide. Furthermore, aspartyl and glutamyl residues are converted to asparaginyl and glutaminyl residues by reaction with ammonium ions.

### Lipophilic Compounds

A peptide according to the present invention and a lipophilic compound may be conjugated to each other, either directly or by use of a linker. The lipophilic compound may be a natural compound such as a saturated or unsaturated fatty acid, a fatty acid diketone, a terpene, a prostaglandin, a vitamin, a carotenoid or steroid, or a synthetic compound such as an organic acid, an alcohol, an amine and sulphonic acid with one or more alkyl-, aryl-, alkenyl- or other multiple unsaturated compounds. Fatty acids may, for example, include fatty acids selected from arachidonic acid, docohexanoic acid and oleic acid. The conjugation between the variant polypeptide and the lipophilic compound, optionally through a linker may be done according to methods known in the art, e.g. as described by Bodanszky in Peptide Synthesis, John Wiley, New York, 1976 and in WO-A-96/12505.

### Immobilisation on a Solid Phase Substrate

The peptide according to the present invention may be soluble or immobilised on a solid phase substrate. Immobilisation may be achieved either directly or by use of a linker.

When the peptide is immobilised on a solid phase substrate, the substrate may be any surface or support, including one or more of a solid support (e.g., glass such as a glass slide or a coated plate, silica, plastic or derivatized plastic, paramagnetic or non-magnetic metal), a semi-solid support (e.g., a polymeric material, a gel, agarose, or other matrix), and/or a porous support (e.g., a filter, a nylon or nitrocellulose membrane or other membrane). In some embodiments, synthetic polymers can be used as a substrate, including, e.g., latex, polystyrene, polypropylene, polyglycidylmethacrylate, aminated or carboxylated polystyrenes, polyacrylamides, polyamides, polyvinylchlorides, and the like. In one preferred embodiment, the substrate comprises a microtiter immunoassay plate or other surface suitable for use in an ELISA. In another preferred embodiment, the substrate can be Agarose or Sepharose beads used to purify AFPr. In another preferred embodiment, the substrate can be beads made of any of the described materials. In another preferred embodiment, the substrate can be magnetic micro- or nano-particles with the capacity to be coated covalently or non-covalently by the peptides. In yet another preferred embodiment, the substrate can be colloidal gold nano-particles.

The surface of the substrate or support may be planar, curved, spherical, rod-like, pointed, wafer or wafer-like, or any suitable two-dimensional or three-dimensional shape on which the second binding partner may be immobilised, including, e.g., films, beads or microbeads, tubes or microtubes, wells or microtiter plate wells, microfibers, capillaries, a tissue culture dish, magnetic particles, pegs, pins, pin heads, strips, chips prepared by photolithography, etc. In some embodiments, the surface is UV-analyzable, e.g., UV-transparent.

Immobilisation may be achieved in any number of ways, known in the art, described herein, and/or as can be developed. For example, immobilisation may involve any technique resulting in direct and/or indirect association of a peptide of this invention (and its corresponding binding protein, AFPr) with the substrate, including any means that at least temporarily prevents or hinders its release into a surrounding solution or other medium. The means can be by covalent bonding, non-covalent bonding, ionic bonding, electrostatic interactions, Hydrogen bonding, van der Waals forces, hydrophobic bonding, or a combination thereof. For example, immobilisation can be mediated by chemical reaction where the substrate contains an active chemical group that forms a covalent bond with the second binding partner. For example, an aldehyde-modified support surface can react with amino groups in protein receptors; or amino-based support surface can react with oxidization-activated carbohydrate moieties in glycoprotein receptors; a support surface containing hydroxyl groups can react with bifunctional chemical reagents, such as N,N-dissuccinimidyl carbonate (DSC), or N-hydroxysuccinimidyl chloroformate, to activate the hydroxyl groups and react with amino-containing receptors. In some embodiments, support surface of the substrate may comprise animated or carboxylated polystyrenes; polyacrylamides; polyamines; polyvinylchlorides, and the like. In still some embodiments, immobilization may utilize one or more binding-pairs to bind or otherwise attach a receptor to a substrate, including, but not limited to, an antigen-antibody binding pair, hapten/anti-hapten systems, a avidin-biotin binding pair; a streptavidin-biotin binding pair, a folic acid/folate binding pair; photoactivated coupling molecules. In a preferred embodiment, the peptide of this invention can be immobilized on Sepharose activated by cyanogen bromide. This is a simple, mild and often successful method of wide applicability. Sepharose is a commercially available beaded polymer which is highly hydrophilic and generally inert to microbiological attack. Chemically it is an agarose (poly-{b-1,3-D-galactose-a-1,4-(3,6-anhydro)-L-galactose}) gel. The hydroxyl groups of this polysaccharide combine with cyanogen bromide to give the reactive cyclic imido-carbonate. This reacts with primary amino groups (i.e. mainly lysine residues) on the enzyme under mildly basic conditions (pH 9-11.5) (Immobilized Affinity Ligand Techniques. Greg T. Hermanson, A. Krishna Mallia and Paul K. Smith. Academic Press,© 1992).

For example, the peptide may be immobilized via hydrogen bonding or hydrophobic interactions by dissolving the peptide in water or a biological buffer, and coating onto a standard plastic plate such as is used for ELISA.

Fragment #4 for example has been covalently linked to a support matrix via cyanogen bromide activation, and coupling via a peptide bond to the N terminal of the peptide. This linkage of the peptide to a matrix allows the peptide to be used as a ligand for the affinity purification of the (AFP) receptor.

### Peptide Aggregates or Polymeric Peptide Matrices

In one embodiment of this aspect of this invention, the peptide may be part of a peptide aggregate or polymeric peptide matrix comprising more than one peptide portions. The peptide portions in the molecule may all be the same as each other, or different peptides may be present as peptide portions. Where different peptides are present, there may be repetition of peptide units (monomers) within the molecule, and the repetition may be regular or irregular.

The peptide portions may be aggregated or conjugated together in a number of ways which are well known in the art, including entanglement or cross-linkage. In another embodiment, the peptide can be polymerized via branched lysine(s) through F-moc chemistry. The peptide through its N-terminal and C-terminal can be linked through standard F-moc chemistry to make conjugates of the peptide.

### Labelling of the Peptides

The peptide according to the present invention may be labelled with one or more detectable label. The label may be conjugated to the peptide either directly or by use of a linker.

One embodiment of this invention provides a labeled AFPr binding peptide wherein a peptide of the invention is derivatized or linked (by chemical coupling, genetic fusion, noncovalent association or otherwise) to another functional molecule to facilitate detection of the bound or unbound peptide.

Where the peptide according to the present invention is conjugated to a direct label, the direct label is suitably an entity which is detectable in its natural state. For example, where the direct label is a coloured particle, such as dye sols, metallic sols (e.g. colloidal gold), and coloured latex particles, this may be visible to the naked eye, or become visible with the aid of an optical filter. Where the direct label is a fluorescent label, this may be subjected to applied stimulation, e.g. UV light to promote fluorescence.

Suitable detectable substances include various enzymes, prosthetic groups, fluorescent materials, luminescent materials and radioactive materials. Exemplary fluorescent detectable agents include fluorescein, fluorescein isothiocyanate, rhodamine, 5-dimethylamine-1-napthalenesulfonyl chloride, phycoerythrin and the like. A peptide may also be derivatized with detectable enzymes, such as alkaline phosphatase, horseradish peroxidase, glucose oxidase and the like. A peptide may also be derivatized with biotin, and detected through indirect measurement of avidin or streptavidin binding.

An example of a luminescent material includes luminol; and examples of suitable radioactive material include ³H, ¹⁴C, ³⁵S, ⁹⁰Y, ⁹⁹TC, ¹¹¹In, ¹²⁵I, ¹³¹I, ¹⁷⁷Lu, ¹⁶⁶Ho, or ¹⁵³Sm. Means of detecting such labels are well known to those of skill in the art. Radiolabels may be detected using photographic film or scintillation counters, fluorescent markers may be detected using a photo detector to detect emitted illumination. Enzymatic labels are typically detected by providing the enzyme with a substrate and detecting the reaction product produced by the action of the enzyme on the substrate. For example, when the detectable agent horseradish peroxidase (HRP) is present the addition of hydrogen peroxide and diaminobenzidine leads to a colored reaction product, which is detectable.

The antibody can be coupled directly with ELISA base labels such as HRP or alkaline phosphatise (AP). The colorimetric signal is then generated by the addition of the appropriate substrate. Labeling of the antibody with biotin also allows either streptavidin-(HRP) or Stretptavidin-(AP) as a secondary reporting molecule. The antibody can also be used in a chemiluminescence assay with the use of acridinium directly or indirectly. In using the antibody in an assay the solid phase is coated with the peptide and the labelled antibody competes for binding onto the peptide with AFP receptor that has come from the sample in the assay.

Conjugation with Detection, Diagnostic and Therapeutic Agents, and Use in Disease Detection, Diagnosis and Treatment

In one embodiment of this aspect of the invention, the peptide according to the invention may be conjugated to one or more detection, diagnostic or therapeutic agent for selective delivery to disease sites such as cancer cells, particularly in humans. The conjugation may be either directly or by use of a linker, analogously to the conjugation described above. The corresponding methods of detection, diagnosis and therapy of the diseases are corresponding aspects of the present invention, as are the peptides for use in such detection, diagnosis and therapeutic methods.

Detection, diagnostic and therapeutic agents may be naturally-occurring, modified, or synthetic. Therapeutic agents may promote or inhibit any biological process implicated in a human disease pathway. The methods of detection, diagnosis and therapy, using the peptides according to the present invention conjugated to one or more detection, diagnostic or therapeutic agent for selective delivery to disease sites, may be performed in vitro, particularly on a sample obtained from a subject, or in vivo in the body of a subject (patient) to be tested or treated.

For example, the additional agent can be a therapeutic agent art-recognized as being useful to treat cancer. The specific binding interaction between the peptide and the cells enables the therapeutic agent to be accurately targeted to cancerous cells in a mammalian patient (after the natural embryonic expression of AFPr has ceased after birth—after birth, serum AFPr is normally at a low background level unless the mammal has a developing or mature cancer, in which case the serum AFPr rises markedly due to expression and release of AFPr by the cancer cells; see WO-A-09551). Therapeutic agents suitable for this use may include any compound that induces apoptosis, cell death, cell differentiation, cell stasis and/or anti-angiogenesis or otherwise affects the survival and/or growth rate of a cancer cell.

In certain embodiments the present invention may concern administration of targeting peptides attached to cytotoxic agents. A wide variety of anti-cancer agents are well known in the art and any such agent may be coupled to a cancer targeting peptide for use within the scope of the present invention. Exemplary cancer chemotherapeutic (cytotoxic) agents of potential use include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of (DTIC), transplatinum, vinblastine and methotrexate, vincristine, or any analog or derivative variant of the foregoing. Most chemotherapeutic agents fall into the categories of alkylating agents, antimetabolites, antitumor antibiotics, corticosteroid hormones, mitotic inhibitors, and nitrosoureas, hormone agents, miscellaneous agents, and any analog or derivative variant thereof.

The peptides of the present invention may be linked to compounds which induce cell differentiation, such as, for example, retinoic acid.

In certain embodiments the present invention may concern administration of targeting peptides attached to anti-angiogenic agents, such as angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP-470, endostatin, paclitaxel, accutin, angiostatin, cidofovir, vincristine, bleomycin, AGM-1470, platelet factor 4 or minocycline. Proliferation of tumors cells relies heavily on extensive tumor vascularization, which accompanies cancer progression. Thus, inhibition of new blood vessel formation with anti-angiogenic agents and targeted destruction of existing blood vessels have been introduced as an effective and relatively non-toxic approach to tumor treatment. (Arap et al., Science 279: 377-380, 1998a; Arap et al., Curr. Opin. Oncol. 10: 560-565, 1998b; Ellerby et al., Nature Med. 5:1032-1038, 1999). A variety of anti-angiogenic agents and/or blood vessel inhibitors are known. (E.g., Folkman, In: Cancer: Principles and Practice, eds. DeVita et al., pp. 3075-3085, Lippincott-Raven, New York, 1997; Eliceiri and Cheresh, Curr. Opin. Cell. Biol. 13, 563-568, 2001).

In certain embodiments of this aspect of the present invention, the peptide of this invention can be linked to alkylating agents; which are drugs that directly interact with genomic DNA to prevent cells from proliferating. This category of chemotherapeutic drugs represents agents that affect all phases of the cell cycle, that is, they are not phase-specific. An alkylating agent, may include, but is not limited to, nitrogen mustard, ethylenimene, methylmelamine, alkyl sulfonate, nitrosourea or triazines. They include but are not limited to: busulfan, chlorambucil, cisplatin, cyclophosphamide (cytoxan), dacarbazine, ifosfamide, mechlorethamine (mustargen), and melphalan.

Apoptosis, or programmed cell death, is an essential process for normal embryonic development, maintaining homeostasis in adult tissues, and suppressing carcinogenesis. Non-limiting examples of pro-apoptosis agents contemplated within the scope of the present invention include gramicidin, magainin, mellitin, defensin, and cecropin.

In other embodiments of this aspect of the present invention, the peptide of this invention can be linked to antimetabolites. Antimetabolites disrupt DNA and RNA synthesis. Unlike alkylating agents, they specifically influence the cell cycle during S phase. Antimetabolites can be differentiated into various categories, such as folic acid analogs, pyrimidine analogs and purine analogs and related inhibitory compounds. Antimetabolites include but are not limited to, 5-fluorouracil (5-FU), cytarabine (Ara-C), fludarabine, gemcitabine, and methotrexate.

In one embodiment, the peptide according to the first aspect of the present invention may include one or more interference oligo- or poly-nucleotides such as iRNA or siRNA. These interference oligo- or poly-nucleotides may suitably be conjugated to the peptide molecule by covalent linkages in conventional manner.

In a different embodiment, the peptide of the present invention can be linked to natural products, a term which generally refer to compounds originally isolated from a natural source, and identified as having a pharmacological activity. Such compounds, analogs and derivatives thereof may be, isolated from a natural source, chemically synthesized or recombinantly produced by any technique known to those of skill in the art. Natural products include such categories as mitotic inhibitors, antitumor antibiotics, enzymes and biological response modifiers. Mitotic inhibitors include plant alkaloids and other natural agents that can inhibit either protein synthesis required for cell division or mitosis. They operate during a specific phase during the cell cycle. Mitotic inhibitors include, for example, docetaxel, etoposide (VP16), teniposide, paclitaxel, taxol, vinblastine, vincristine, and vinorelbine. Taxoids are a class of related compounds isolated from the bark of the ash tree, Taxus brevifolia. Taxoids include but are not limited to compounds such as docetaxel and paclitaxel. Paclitaxel binds to tubulin (at a site distinct from that used by the vinca alkaloids) and promotes the assembly of microtubules.

In another embodiment of this aspect of this invention, the peptide of the present invention can be linked to antibiotics. Certain antibiotics have both antimicrobial and cytotoxic activity. These drugs also interfere with DNA by chemically inhibiting enzymes and mitosis or altering cellular membranes. These agents are not phase specific so they work in all phases of the cell cycle. Examples of cytotoxic antibiotics include, but are not limited to, bleomycin, dactinomycin, daunorubicin, doxorubicin (Adriamycin); plicamycin (mithramycin) and idarubicin.

In other embodiments of this invention, the peptide of the present invention can be linked to miscellaneous therapeutic agents. Miscellaneous cytotoxic agents that do not fall into the previous categories include, but are not limited to, platinum coordination complexes, anthracenediones, substituted ureas, methyl hydrazine derivatives, amsacrine, L-asparaginase, and tretinoin. Platinum coordination complexes include such compounds as carboplatin and cisplatin (cis-DDP). An exemplary anthracenedione is mitoxantrone. An exemplary substituted urea is hydroxyurea. An exemplary methyl hydrazine derivative is procarbazine (N-methylhydrazine, MIH). These examples are not limiting and it is contemplated that any known cytotoxic, cytostatic or cytocidal agent may be attached to targeting peptides and administered to a targeted organ, tissue or cell type within the scope of the invention.

In certain embodiments, it may be desirable to couple specific bioactive agents to one or more targeting peptides for targeted delivery to an organ, tissue or cell type. Such agents include, but are not limited to, cytokines and/or chemokines. The term “cytokine” is a generic term for proteins released by one cell population that act on another cell as intercellular mediators. Examples of cytokines are lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1.alpha., IL-2, IL-3, IL-4, IL-5, IL-6, IL-7, IL-8, IL-9, IL-10, IL-11, IL-12; IL-13, IL-14, IL-15, IL-16, IL-17, IL-18, LIP, G-CSF, GM-CSF, M-CSF, EPO, kit-ligand or FLT-3, angiostatin, thrombospondin, endostatin, tumor necrosis factor and LT. As used herein, the term cytokine includes proteins from natural sources or from recombinant cell culture and biologically active equivalents of the native sequence cytokines. Chemokines generally act as chemo-attractants to recruit immune effector cells to the site of chemokine expression. It may be advantageous to express a particular chemokine gene in combination with, for example, a cytokine gene, to enhance the recruitment of other immune system components to the site of treatment. Chemokines include, but are not limited to, RANTES, MCAF, MIP1-alpha, MIP1-Beta, and IP-10. The skilled artisan will recognize that certain cytokines are also known to have chemoattractant effects and could also be classified under the term chemokines.

### Liposomes

The peptide according to the present invention may be present in liposomal form or conjugated to a liposome or conjugated to a lipid capable of forming a liposome. The lipid may, for example, be a phospholipid or a fatty acid. A suitable phospholipid may, for example, be phosphatidyl choline, phosphatidyl glycerol, phosphatidyl inositol, any myristylated or stearylated or oleylated derivative thereof, or any combination thereof. A suitable fatty acid may, for example, be arachidonic acid, docohexanoic acid, oleic acid, or any combination thereof. The peptide may suitably be present externally of the liposome. The liposome may suitably encapsulate, or present on its surface, bioactive agents or other molecules, for example labels for imaging (for example radiolabels for imaging), therapeutic agents (for example radioactive isotopes for radiotherapy of cancer), drug molecules for therapy, enzymes, toxins, interference oligo- or poly-nucleotides, viruses, specific binding molecules, for example antibodies, having specific binding capacity with moieties, for example antigens (for example AFPr), presented on the surface of cells, for example cancerous cells.

### Oncolytic Viruses to AFPr-Expressing Cancer Cells

In one embodiment of the present invention, the peptide according to the present invention may be conjugated to an oncolytic virus. Such an embodiment can be used to target oncolytic viruses to cancer cells expressing AFPr. The term “oncolytic” refers to lysis or breakdown of cancer cells through the process of apoptosis. The oncolytic virus itself can destroy tumor cells by replicating. This cycle then can repeat, by infection of adjacent cells and their subsequent destruction by the same mechanism. This feature of viral replication provides continuous amplification of the input dose which continues until stopped by the immune response or a lack of susceptible cells (John et al., The Oncologist 7: 106-119, 2002).

A second mechanism used by oncolytic viruses is the syntheses of proteins during replication that are directly cytotoxic to cancer cells. For example, adenoviruses generate the death protein E3 11.6 kD and the E4ORF4 protein late in the cell cycle; both these proteins are toxic to cell (Tollefson et al. Virology 220:152-162; Shtrichman et al. J Virol; 72:2975-2982. 1998).

A third mechanism by which oncolytic viruses act is by initiating specific and nonspecific anti-tumor immune responses. In addition, viral infection can induce specific anti-tumor immunity. Tumor cells are inherently weakly immunogenic because they express low levels of major histocompatibility complex antigens and stimulatory signals such as cytokines which activate a local immune response. For example, adenoviruses express E1A protein during replication, which mediates killing of tumor cells by increasing their sensitivity to tumor necrosis factor (TNF) (Gooding et al. Infect. Agents Dis; 3:106-115, 1994). Induction of specific anti-tumor immunity might result in long-term defense against cancer recurrence. Viral peptides are presented on the cell surface with MHC class I proteins; this complex is recognized by cytotoxic T lymphocytes (CTLs), which are attracted to the virally-transduced tumor cell. These CTLs then acquire specificity for cancer-specific antigens and kill the cells by a still unknown mechanism (Toda et al. Human Gene Therapy; 10:385-393, 1999).

In a preferred embodiment, an oncolytic virus, for example an adenovirus, will be genetically engineered to express a peptide according to the present invention on its coat, thus increasing entry into target AFPr-expressing cells while reducing entry to non-target cells. To avoid inactivation of circulating viruses, the virus can be formulated in PEG, liposome or collagen matrices or in combination with immune suppressants.

### CD8+ and CD4+ T-cell Target Peptides and Vectors Therefore

CD8+ T cells (TCD8+) are important effectors in antiviral immunity (Kaech, S. M., E. J. Wherry, and R. Ahmed. 2002. Effector and memory T-cell differentiation: implications for vaccine development. Nat. Rev. Immunol. 2:251-262; Yap, K. L., G. L. Ada, and I. F. McKenzie. 1978. Transfer of specific cytotoxic T lymphocytes protects mice inoculated with influenza virus. Nature 273: 238-239.), recognizing virus peptides presented on infected cells by major histocompatibility complex (MHC) class I (Zinkernagel, R. M., and P. C. Doherty. 1979. MHC-restricted cytotoxic T cells: studies on the biological role of polymorphic major transplantation antigens determining T-cell restriction-specificity, function, and responsiveness. Adv. Immunol. 27:51-177).

The general mechanism of cytotoxicity involves the expression of a viral peptide in the groove of the Class I histocompatibility complex which is recognized by CD8+ T cells that destroy the infected cell.

Since CD4+ T cells also contribute to the immune response, the AFP fragment can also transport longer peptide fragments, proteins that would cleaved into peptides inside the cell or nucleic acids that produce peptides that upon fragmentation inside the cell are expressed in the Class II complex.

The peptide according to the present invention may be conjugated to such a peptide, to a larger peptide that is a precursor for the CD8+ or CD4+ T cell recognized peptide, or to a vector that induces cell surface expression of the peptide or precursor as part of the Class I or Class II complex on an infected, particularly cancerous, cell.

The peptide according to the present invention can thus be used to introduce any of these materials selectively into cancer cells (i.e. avoiding the subsequent destruction of normal cells not expressing the AFP receptor).

As an example of this application, a cancer patient would be subjected to an immunization/infection (i.e. by inoculating him or her with vaccinia, influenza, or other virus inducing a strong T cell response). Once the acute phase of the infection is over, the patient would be injected with a conjugate of the peptide according to the present invention any of the materials mentioned above which would lead to establishment of the CD8+ or CD4+ T cell recognized peptide on the cell surface. For example, a short peptide of the vaccinia virus that is commonly expressed in the Class I or Class II complex and which induces cytotoxicity could be attached or simply synthesized as an extension of the AFP fragment, directly or via a spacer designed to break off and release the viral peptide once inside the cell.

The peptides that bind to AFPr according to the present invention can be used to selectively introduce these peptides/proteins into cancer cells because these cancer cells express AFPr. Normal cells that do not express AFPr would not bind the peptide and thus would not process the peptides/proteins associated with AFP. Following interaction at the cell surface with AFPr, the AFP peptide is internalized and any peptides/proteins also transported with the AFP peptide are processed by MHC class I processing pathways with an ultimate presentation at the cell surface in the context of the MHC class I molecule. Thus cells expressing AFPr can be manipulated to express a specific peptide presented by the MHC class I molecule thus becoming a target for peptide-specific CD8+ T cells. The presence of the peptide specific CD8+ cytotoxic T cells will cause the destruction of cells expressing the targeted peptide.

### Assays

The peptide according to the present invention may be used in a method for the detection (including quantitative detection in an assay method) of AFPr.

In a method for detection of AFPr, the peptide according to the first or third aspect of the present invention is contacted with a sample containing or suspected to contain AFPR, under conditions permitting binding or the peptide to the AFPr, and binding of the peptide to any AFPr present is subsequently detected.

The detection of the AFPr may suitably be quantitative.

One or more of the reagents may be labeled for detection. For example, the peptide according to the first or third aspect of the present invention may be labeled. Alternatively or additionally, the peptide according to the first or third aspect of the present invention may compete in the assay with another specific binding partner of AFPr, for example AFP, one or more of the competing entities being labeled for detection. Alternatively or additionally, the peptide according to the first aspect of the present invention may be contacted with the sample in the presence of labeled AFPr, whereby the labeled and unlabelled AFPr compete for binding with the peptide.

The sample is suitably a sample of a body fluid or tissue taken from a human or animal patient. The body fluid may be blood, serum, plasma, mucus, urine, faeces, sputum, saliva or tears. The body tissue may be cancer tissue or other material containing cancer cells, for example tissue obtained by biopsy. A tissue section obtained from a tissue sample may be used. The sample may contain cells in suspension, for example a cervical smear.

The cancer cells may suitably be cancerous or tumorous cells obtained from tissue and body fluids selected from ovary, lymph node, blood, limb, soft tissue, skin, stomach, intestine, breast, abdomen, uterus, cervix, bladder, prostate, rectum, colon, pelvis, brain, lung, liver, kidney or bone. As described in WO-A-96/09551, the detection of AFPr can reveal cancers in these and other tissues, such as adenocarcinoma, leukemia, agiosarcoma, sarcoma, carcinomatosis, generally spread tumors, astrocytoma, osteosarcoma, epithelioma, and primary and metastatic neoplasia, for example in or originating from the ovary, lymph node, blood, limb, soft tissue, skin, stomach, intestine, abdomen, uterus, cervix, bladder, rectum, bone, brain, lung, liver, pelvis, prostate, breast, kidney and colon.

A benign tumor or an inflammatory lesion may alternatively be detected according to the method of the present invention for detecting the presence or absence of AFPr in a patient or a biological sample obtained from the patient.

The peptide is suitably labelled for detection and may be immobilised on a solid support. The peptide serves as the specific binding partner for any AFPr in the sample. After the binding of the peptide or antibody to the AFPr is complete, labelled material that has not participated in a binding reaction is generally removed and the pattern of labelling of the material that has participated in a binding reaction is assayed by detection of the label. Normally, the amount of label associated with the product of the binding reaction is quantitated.

In one embodiment the peptide is unlabelled and is immobilised on a solid support. The sample to be tested is contacted with the immobilised unlabelled peptide in the presence of labelled AFPr which competes with any AFPr in the sample for binding to the peptide or antibody. The binding reaction is allowed to go to completion under suitable conditions, after which the immobilised phase of the system is removed, washed, and the presence of label incorporated in the immobilised phase is detected by a suitable detection method for the label (suitably, a quantitative detection method). This detection system is known as a competition assay. It is preferred according to the present invention, because it is only necessary for the peptide or antibody to have one potential binding site on the AFPr. Provided that the labelled AFPr is labelled in a manner that does not interfere with the binding interaction, the ability of the AFPr in the sample and the labelled AFPr to bind with the immobilised peptide or antibody will correspond, and the extent of incorporation of the label into the immobilised phase will be in inverse proportion to the concentration of AFPr, in the sample.

Any suitable label may be used on the peptide or the competing AFPr. Examples of labels, and their detection systems, that can be used on the peptide or antibody have been described above.

In one embodiment of the invention a method to detect AFPr may use a suitable support (e.g. a membrane) coated with one or more peptide according to the present invention, e.g. in a band on the support. The sample to be assayed or tested for the presence or absence of AFPr can be mixed with AFPr labelled with a suitable label (such as, for example but not limited to, colloidal gold, iron particles, iron nanoparticles, colored latex particles and fluorophores) and thoroughly contacted with the immobilised peptide according to the invention (e.g. by being subjected to a lateral flow chromatography in the support membrane).

A further embodiment of the method of the present invention to detect AFPr is the so-called “sandwich” assay, wherein the support is coated with one or more specific binding partner for AFPr. After contacting the immobilised specific binding partner for AFPr with the sample suspected of containing AFPr, and removal of unbound AFPr, the resultant immobilised AFPr can be contacted with a labelled species capable of specifically binding to the immobilised AFPr, followed by removal of unbound label, whereby on detection of the label (suitably, quantitatively), the concentration of AFPr in the sample can be determined. According to the present invention, at least one of the immobilised and the labelled species capable of specifically binding to AFPr in the sample should be a material according to the first or third aspects of the present invention.

### Diagnosing Cancer

The assay method described above may be used, after setting a suitable threshold concentration of AFPr that is, with acceptable sensitivity and specificity, diagnostic of cancer in a method of diagnosing cancer in a human or non-human mammalian patient. For further details see WO-A-96/09551.

Visualization, Imaging and Audio Detection of Cancer Cells or other AFPr-Expressing Lesion

The labelled peptide according to the present invention will bind to AFPr expressed by cancer cells or other AFPr-expressing lesion such as a benign tumor or an inflammatory lesion. By using a label that is visible under a suitable microscope, the presence of cancerous tissue or cells, or other AFPr-expressing lesion, in a biological sample obtained from a patient can be visualized, imaged or auditorily detected in essentially the same way as previously described in relation to labelled anti-AFPr antibodies. For further details see WO-A-96/09551 and R. Moro et al., “Monoclonal antibodies against a widespread oncofetal antigen: the alpha-fetoprotein receptor”, Tumor Immunology, vol. 14, no. 2, 1 Jul. 1993, pages 116-130).

The invention thus also enables a method of in vivo detection and/or imaging of cancer or other AFPr-expressing lesion. The peptide of the present invention, suitably labeled for in vivo visualisation, can be administered to a subject and the extent of retention of the label to tissues and organs of the subject's body can be monitored by monitoring the distribution of the label. Suitable labels for this purpose include radioactive isotopes, the distribution of which in the subject's body can be monitored using a suitable instrument, for example a gamma counter, a gamma scanner, a Geiger counter or hand-held radioactivity detector) to detect the radioactivity after a suitable time period. For example, a gamma camera or scanner can be used for imaging cancers or other AFPr-expressing lesions within the subject's body. A Geiger counter or any hand-held, e.g. wand type, radioactivity detector can be used in surgery where the tumors might be located by moving the wand inside the surgical theater and detecting the tumoral masses by the audible noises emitted by the instrument.

### Testing for Pregnancy

The assay method described above for detecting whether a biological sample contains AFPr can be used to detect pregnancy in a female human or animal, after setting a suitable threshold concentration of AFPr that is, with acceptable sensitivity and specificity, diagnostic of pregnancy in a female human or animal. For further details see WO-A-96/09551.

### Purification of AFPr

In a fourth aspect the present invention provides a method for purifying AFPr which comprises binding said AFPr to the material (peptide, including antibody) according to the first or third aspect of the invention. The peptide/AFPr complex that results from this binding interaction can then be separated from the mixture. The AFPr can then be obtained from the complex in relatively pure form.

In the method the AFPr or cells expressing the same are contacted with the peptide according to the present invention under conditions permitting binding of the peptide to the AFPr or cells, unbound peptide is removed, the peptide is unbound from any AFPr or cells (see WO-A-96/09551) and the AFPr or cells recovered in a relatively pure state.

In one embodiment the peptide or antibody is immobilised on a solid support. As large a concentration of such immobilised material as possible is preferably present, and the nature of the immobilised phase, for example the shape of the surface and the concentration of the peptide per unit area, is preferably chosen to maximise the access to the immobilised phase by the AFPr. The material to be purified is contacted with the immobilised peptide. The binding reaction is allowed to go to completion under suitable conditions, after which the immobilised phase of the system is removed, washed, and the AFPr or cells incorporated in the immobilised phase are removed by unbinding. The purified AFPr or cells is then recovered, optionally after further purification (e.g. repeated purification).

Each aspect of the present invention and each embodiment of any aspect of the present invention may be present or used independently of any other aspect of the invention or embodiment thereof, or may be present or used together with one or more of such other embodiments and/or aspects, as desired.

### BRIEF DESCRIPTION OF THE DRAWINGS

In the accompanying drawings:

FIG. 1 shows the 12% SDS Page gel patterns in the AFP digestion experiment of Example 1;

FIG. 2 shows the amino acid sequence of human AFP as published in Uniprot database;

FIG. 3 shows the results of the inhibition experiment described in Example 3;

FIG. 4 shows the tabulated results from the repetitions of Example 3 described in relation to different peptides;

FIG. 5 shows the data obtained in Example 2, relating to the experiment for detection of AFPr using Fragment #4;

FIG. 6 shows the 10% SDS Page gel of AFPr purified by the Fragment #4 column as described in Example 2;

FIG. 7 shows the result of using AFPr, purified as described in Example 2 and subsequently radiolabelled, to test 237 miscellaneous cancer and normal samples for presence or absence of cancer, via a competition immunoassay to assay AFPr expression using 1.4G11 anti-AFPr monoclonal antibodies;

FIG. 8 shows the binding of FITC labelled Fragment #4 to cancer cells, as described in Example 2;

FIG. 9 shows the use of Fragment #4 to capture AFPr in an assay using acridinium labelled 1.4G11 anti-AFPr monoclonal antibodies; and

FIG. 10 shows uptake of FITC labelled Fragment #4 into MCF-7 human breast cancer cells.

### EXAMPLES AND DETAILED DESCRIPTION OF THE DRAWINGS

The following non-limiting Examples provide further description of the present invention, with reference to the drawings.

### Example 1

### Determination of AFPr Binding Site in AFP

Specific biological activity of AFP has been previously demonstrated in proteolytic fragments (Dudich et al. (1999) Biochemistry, 38: 10406-10414). The same approach, namely a proteolytic digest, was used to determine the binding site for the alpha-fetoprotein receptor.

A series of enzymatic digests were performed with whole human AFP. The source for the AFP was the human cell line HepG2 which secretes AFP and is comparable to AFP from human origin (Deutsch et al. 2000 Tumour Biol; 21: 267-277).

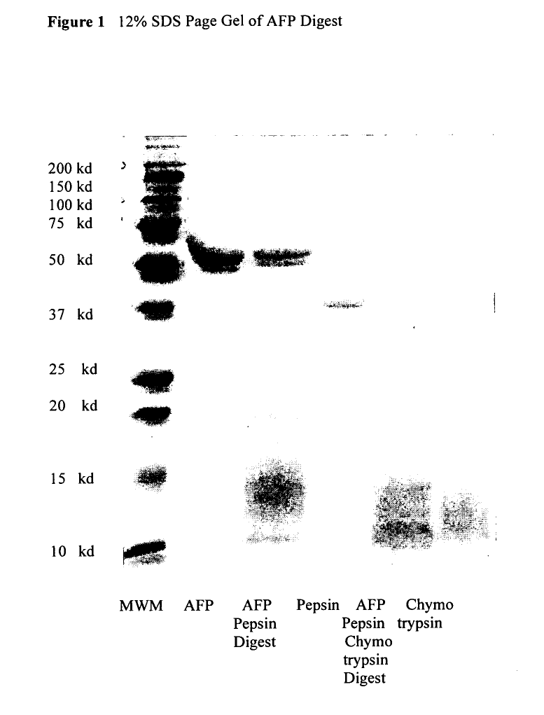

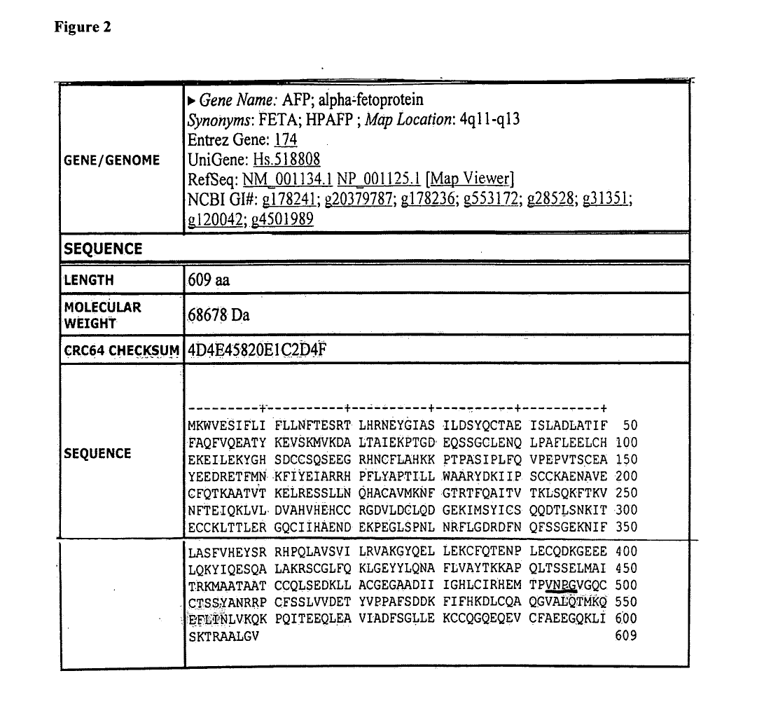

To see if the enzymatic digests were still biologically active, the enzymatic digests were used in a competitive based ELISA with whole AFP molecule labeled with biotin. The ability of the enzymatic digest dilutions to compete for the alpha-fetoprotein receptor present in MCF-7 cell extracts was determined using a constant 0.5 ug/ml of biotin labeled AFP. The smallest fragment generated which still competed was a fragment from a pepsin followed by a chymotrypsin digest (FIG. 1) which yielded a fragment of 10 to 15 KDa in size visualized using a 12% SDS Page gel. This fragment was run on a gradient tricine gel, transferred to a PVDF membrane and sent for N terminal sequencing. Results gave the amino acid (AA) sequence V-N-P-G . . . corresponding to AA 493 to 496 of Human AFP as published in Uniprot Data base (FIG. 2).

Biological function has been shown from synthetic peptides synthesized from the AFP sequence. (Mesfin et al., (2000) Biochim. Biophys. Acta, 1501:33-34; Mizejewsky, et al., (1996) Mol. Cell. Endocr., 118: 15-23. Fragments from AA 493 to 554 in human AFP were synthesized in segments less than 23 AA long using standard Fmoc chemistry. From the C terminal AA 555 to 609 had already been previously synthesized and did not compete. The synthesized fragment, 22 AA long with the sequence: HKDLCQAQGVALQTMKQEFLIN (SEQ. ID. NO: 5) (Fragment 3) was found to compete with AFP biotin labeled. An alternative fragment HKDKDLCQAQGVALQTMKQEFLIN (SEQ. ID. NO: 17) (also referred to as Fragment 3 in the US patent application from which this application claims priority) was mentioned in that US application as having a similar competing activity. To see if the sequence could be further reduced the sequence was reduced to two 11 amino acid sequences. The sequence LQTMKQEFLIN (SEQ. ID. NO: 6) (Fragment 4) was found to still compete. Additional peptides where synthesized and the smallest fragment found to compete was KQEFLIN (SEQ. ID. NO: 3). To see if another species corresponding AFP sequence peptide fragment would also compete with the receptor. The mouse AFP peptide fragment comparable to human was also synthesized. In this fragment the phenylalanine was substituted for leucine. This fragment was also found to compete with whole human AFP for the binding of the receptor.

### Example 2

### Detection and Purification of AFP Receptor, and Visualization of Cancer

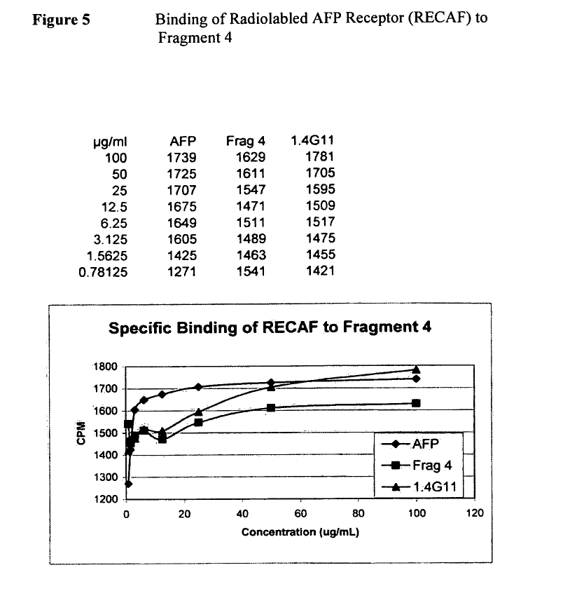

To show directly that the peptide Fragment 4 does bind the AFP receptor (AFPr or RECAF), Fragment 4 was coated onto a plate with an initial concentration of 100 ug/ml and half dilutions made. As controls, whole AFP and the monoclonal antibody 1.4G11 (which is an antibody binding the receptor of AFP; see R. Moro et al., “Monoclonal antibodies against a widespread oncofetal antigen: the alpha-fetoprotein receptor”, Tumor Immunology, vol. 14, no. 2, 1 Jul. 1993, pages 116-130) were also coated. The plate was blocked and incubate with radio-labeled AFP receptor to see if there is binding (FIG. 5).

As whole AFP has been used to purify the receptor via column chromatography using covalently linked AFP, the same procedure was also done using Fragment 4.

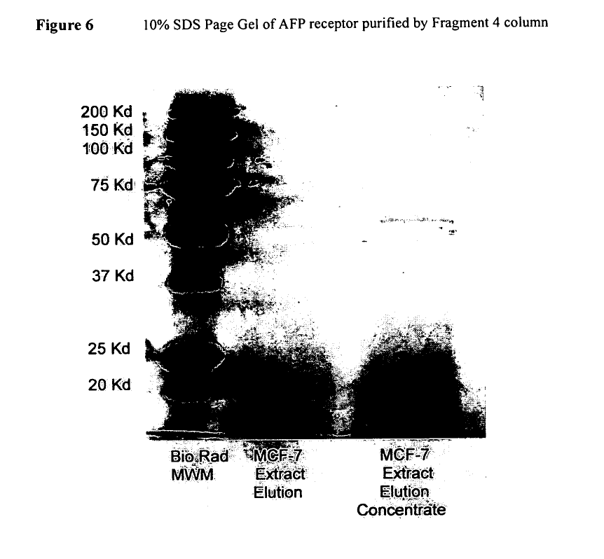

The fragment was coupled to cyanogen bromide activated sepharose 6B. The peptide was coupled at 5 mg per ml of activated sepharose, washed and equilibrated with the same buffer as is used to for the competition assay. The peptide column was used to purify RECAF from a cancer cell extract (FIG. 6).

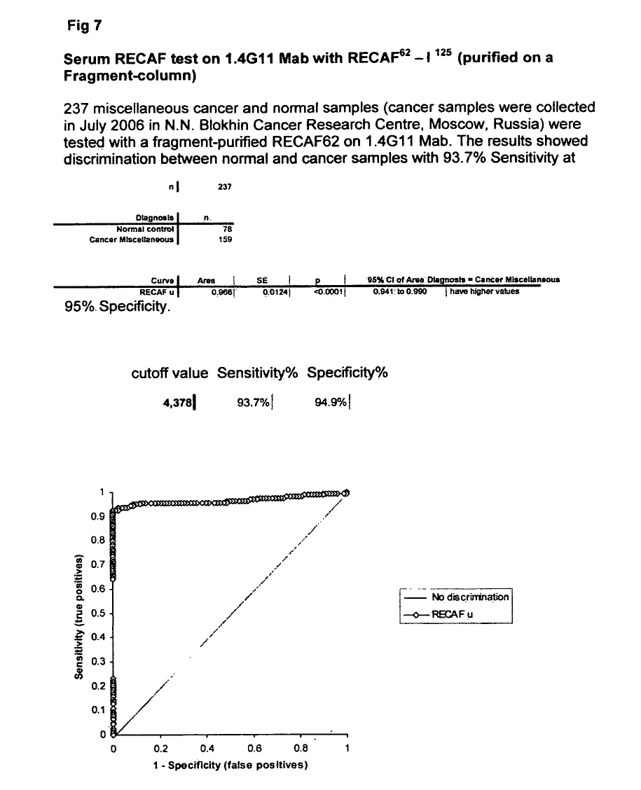

The AFP receptor that was purified from the AFP fragment column was radio-labeled and used to show that the receptor could be used to distinguish cancer from normal samples (FIG. 7). The radiolabelled purified AFPr is denoted as RECAF⁶²-I¹²⁵. 237 miscellaneous cancer and normal samples (the cancer samples were collected in July 2006 in the N.N.Blokhin Cancer Research Centre, Moscow, Russia) were tested in a competition immunoassay using the anti-AFPr monoclonal antibodies (1.4G11 MAb). The results showed discrimination between normal and cancer samples with 93.7% sensitivity and 95% specificity (see FIG. 7).

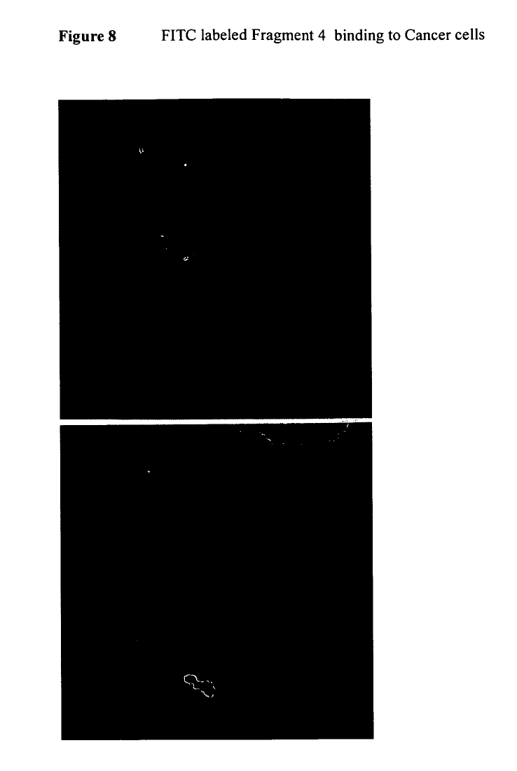

As the peptide Fragment 4 was shown to bind to the AFP receptor when the receptor was radio-labeled, Fragment 4 was synthesized with FITC at the N terminal. The fragments where incubated with a cancer cell line that is known to have AFP receptors, and demonstrated binding to the cancer cells (FIG. 8).

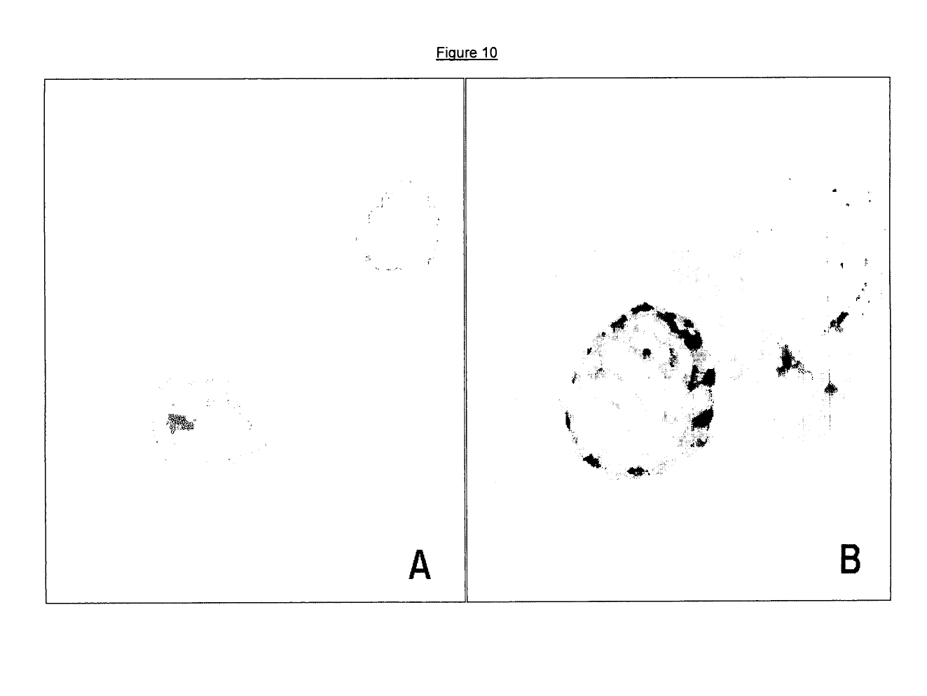

To show that the peptides according to the present invention can be taken up into cancer cells, rather than always binding to surface receptors, we contacted the FITC labeled Fragment 4 peptide with MCF-7 cells (human breast cancer cell line). FIGS. 10A and B show the results of this experiment. FIG. 10A shows the fluorescence from the FITC label. FIG. 10B shows the fluorescence with white lighting to see the shape of the cells. This experiment provides evidence that the peptides according to the present invention provide for intracellular delivery of bioactive agents to the interior of cells expressing AFPr, particularly cancer cells.

### Example 3

### Use in Assay for AFPr

A competition assay with AFP-biotin has been devised to test whether the peptides have utility in an assay for AFPr.

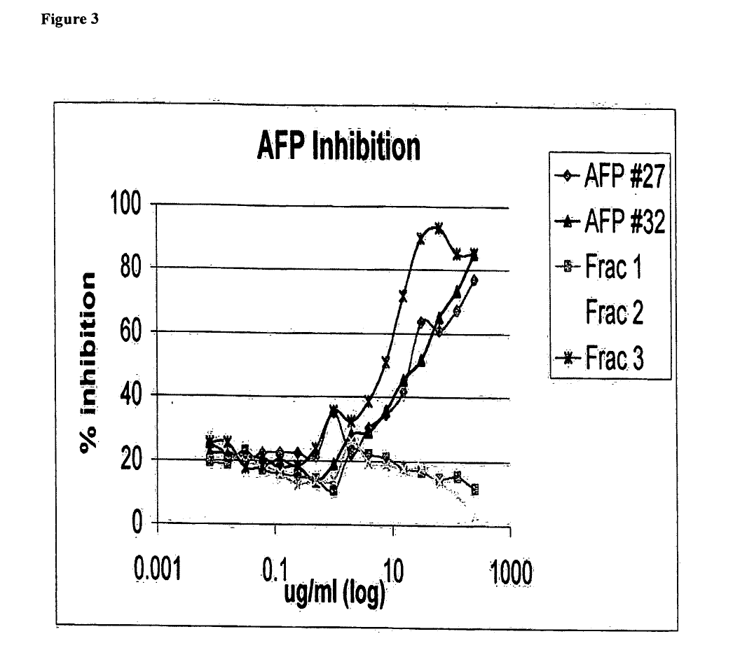

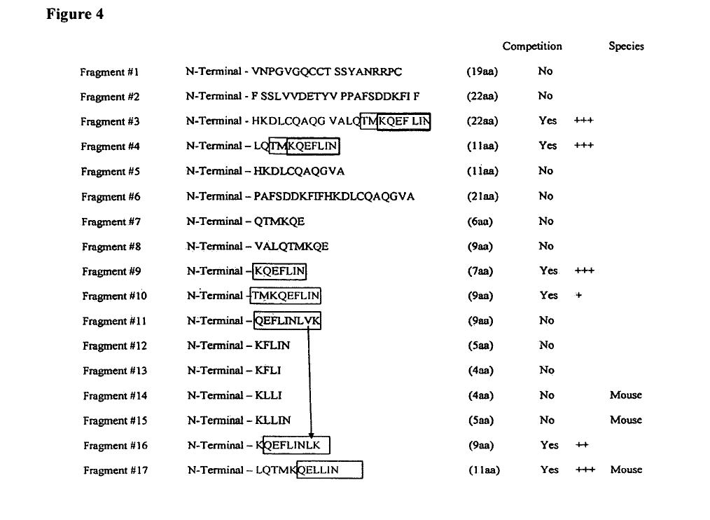

A plate can be coated with an extract of a cancer cell line known to have AFP receptors (for example, MCF-7). The plate is coated at 100 ug/ml total protein concentration and the peptide to be tested and AFP biotin is mixed with an initial peptide concentration of 250 μg/ml and subsequent half dilutions made. The initial 22 AA long peptide HKDLCQAQGVALQTMKQEFLIN (SEQ. ID. NO: 5) (Fragment 3) was synthesized competed with 85% inhibition at 250 μg/ml initially down to 50% inhibition at 7.8 μg/ml with a constant AFP-biotin concentration of 0.5 μg/ml. (FIG. 3). This assay was repeated with subsequent peptides synthesized in order to determine the smallest amino acid chain that would compete along with other peptides not in this region of the AFP molecule as well as from a different species. The results are tabled in (FIG. 4) The 11 amino acid peptide, Fragment 4 is easily synthesized and is soluble without the need for a solvent.

The SEQ. ID. NOS of the other sequences identified in FIG. 4 are as follows:

| SEQ. ID. NO: 18 | |
|---|---|
| Fragment #1 | VNPGVGQCCTSSYANRRPC |
| SEQ. ID. NO: 19 | |
| Fragment #2 | FSSLVVDETYVPPAFSDDKFIF |
| SEQ. ID. NO: 20 | |
| Fragment #5 | HKDLCQAQGVA |
| SEQ. ID. NO: 21 | |
| Fragment #6 | PAFSDDKFIFHKDLCQAQGVA |
| SEQ. ID. NO: 22 | |
| Fragment #7 | QTMKQE |
| SEQ. ID. NO: 23 | |
| Fragment #8 | VALQTMKQE |
| SEQ. ID. NO: 24 | |
| Fragment #11 | QEFLINLVK |
| SEQ. ID. NO: 25 | |
| Fragment #12 | KFLIN |
| SEQ. ID. NO: 26 | |
| Fragment #13 | KFLI |
| SEQ. ID. NO: 27 | |
| Fragment #14 | KLLI |
| SEQ. ID. NO: 28 | |
| Fragment #15 | KLLIN |
| SEQ. ID. NO: 29 | |
| Fragment #16 | KQEFLINLK |

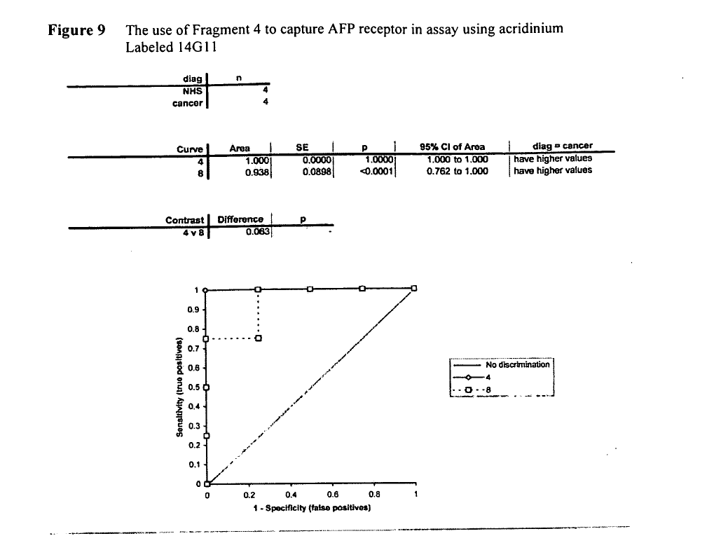

To show that the fragment itself can be used as a replacement for an antibody used in a sandwich assay Fragment 4 was used to coat a plate to capture AFP receptor in 4 cancer and 4 normal samples to show discrimination (FIG. 9).

### INDUSTRIAL APPLICABILITY

The present invention makes available an improved system for assaying and targeting the AFP receptor, useful for assaying biological samples for the presence or absence of cancer, for testing a biological sample for pregnancy, and for targeting anti-cancer and other therapeutic agents to AFPr-positive cells.

The peptide agents used in the present invention may, for example, be prepared synthetically. Labels and bioactive molecules may, for example, be conjugated to the peptide agents for use in the present invention.

The peptide can be used as a substitute for an antibody in a diagnostic format by labeling the peptide directly with a molecule using standard procedures that can generate a signal such as horseradish peroxidase, acridinium, radioisotope, fluorescent molecules, or indirectly with biotin. The peptide has peptide bonds and side groups that can be labeled as would the original AFP molecule or an antibody. As the peptide can be modified by the addition of amino acids, the level and the specificity of the labeling can be controlled directly, which is not available with antibody labeling.

The peptide can be used as a coating for a plate or substrate to replace an antibody or AFP, again the peptide synthesized by standard F-moc chemistry so as to optimize the peptide's ability to act like an antibody and recognize the receptor for AFP.

The peptide itself can be used as a carrier of molecules that can be used to identify or alter a cancer cell by binding to the AFP receptor. The N terminal or the carboxyl terminal of a peptide or modified peptide can be labeled.

Peptides that are water soluble or water insoluble can be selected as desired, allowing for fine adjustment of the assay system, the cancer visualization system, the AFPr purification system, the diagnostic system or the therapeutic system.

The foregoing broadly describes the present invention without limitation. Variations and modifications as will be readily apparent to those skilled in the art are intended to be within the scope of the invention as defined in and by the claims.

The following numbered paragraphs define particular embodiments of the present invention:
1. A peptide containing the sequence Lys-Gln-Glu-Phe-Leu-Ile-Asn (same as SEQ. ID. NO: 3) that binds to the human alpha-fetoprotein (AFP) receptor (AFPr).
2. The peptide in paragraph 1 in which some amino-acids have been substituted as follows:

(same as SEQ. ID. NO : 12)
Lys-Gln-Gln-Phe-Leu-Ile-Asn
(same as SEQ. ID. NO: 13)
Lys-Gln-Gln-Phe-Ile-Asp-Leu
(same as SEQ. ID. NO: 11)
Lys-Glu-Gln-Phe-Leu-Ile-Asn
(same as SEQ. ID. NO: 14)
Lys-Gln-Gln-Leu-Ile-Asp-Leu
(same as SEQ. ID. NO: 9)
Lys-Gln-Glu-Leu-Leu-Ile-Asn
(same as SEQ. ID. NO: 10)
Lys-Glu-Glu-Phe-Leu-Ile-Asn.

3. The peptides in paragraphs 1 and 2 in which some amino-acids have been substituted by similar amino-acids of the same group.
4. The peptides in paragraphs 1-3 to which amino-acids are added on one end of the peptide, the other end or both ends to render the peptide less soluble. This is to enhance adsorption of the peptide to solid phases.
5. The peptides in paragraphs 1-3 to which amino-acids are added on one end of the peptide, the other end or both ends to render the peptide more soluble.
6. The peptides in paragraphs 1-3 to which amino-acids are added on one end of the peptide, the other end or both ends to covalently link them to other molecules.
7. The peptides in paragraphs 1-3 to which one or more cystein molecules are added to form disulphide bonds with other molecules.
8. The peptides in paragraphs 1-3 to which one or more tyrosine molecule or other suitable molecules are added to enable radiolabeling of the peptide.
9. A polymer (or aggregate) of the peptides in paragraphs 1-8, expressing more than one AFPr binding site, in which all the peptide chains are the same.
10. A polymer (or aggregate) of the peptides in paragraphs 1-8, expressing more than one AFPr binding site, in which the peptide chains are different.
11. The materials in paragraphs 1-10 in which the material is labeled with a radioisotope.
12. The materials in paragraphs 1-10 in which the material is labeled with biotin.
13. The materials in paragraphs 1-10 in which the material is attached to a molecule of interfering Ribonucleic Acid (iRNA) or small interfering Ribonucleic Acid (siRNA).
14. The materials in paragraphs 1-10 in which the material is attached to liposomes. These liposomes could be carrying many different things, from radioactivity for imaging and therapy to enzymes to toxins to drugs to iRNA or virus, etc, that allows us to detect or kill cells expressing AFRr.
15. The materials in paragraphs 1-10 in which the material is attached to substances that induce cell differentiation such as, but not limited to retinoic acid.
16. The materials in paragraphs 1-10 in which the material is attached to substances that induce apoptosis.
17. The materials in paragraphs 1-10 in which the material is labeled with a suitable drug or toxin that kills cells expressing AFPr.
18. The materials in paragraph 17 in which the AFPr expressing cells are in-vitro.
19. The materials in paragraph 17 in which the AFPr expressing cells are inside the patient and the materials are introduced into the patient.
20. The materials in paragraphs 1-10 in which the material is attached or aggregated with a fatty acid such as but not limited to arachidonic acid, docohexanoic acid, oleic acid.
21. A method to purify AFPr in which said AFPr binds to the material in paragraphs 1-10.
22. The method described in paragraph 21 in which the material in paragraphs 1-10 is covalently attached to a solid phase.
23. The method described in paragraph 21 in which the material in paragraphs 1-10 is first reacted with material containing AFPr and then the complex is separated from the mixture.
24. A method to detect AFPr in which the material in paragraphs 1-10 is labeled with a suitable label and then incubated with a sample containing AFPr.
25. The method in paragraph 24 in which the sample is a tissue section.
26. The method in paragraph 24 in which the sample is a bodily fluid.
27. The method in paragraph 24 in which the sample contains cells in suspension.
28. A method to detect AFPr in which a suitable support membrane is coated with a band of one of the materials described in paragraphs 1-10 and the sample is mixed with AFPr labeled with a suitable label such as but not limited to colloidal gold, iron particles, iron nanoparticles, colored latex particles and fluorophores and subject to a lateral flow chromatography in the support membrane. This paragraph is to cover use of the peptide as a substitute for the antibody used in rapid tests. The paragraph has to cover sandwich as well as competitive assays. In the case of the sandwich the test can use a combination of one antibody and the materials in paragraphs 1-10 or just the materials in paragraphs 1-10 [in other words, the peptide is on both sides of the sandwich]).
29. The methods described in paragraphs 24, 25, 26, 28 in which the lesion is a malignant tumor or leukemia.
30. The methods described in paragraphs 24, 25, 26, 28 in which the lesion is a benign tumor.
31. The methods described in paragraphs 24, 25, 26, 28 in which the lesion is an inflammatory lesion.
32. The method described in paragraphs 24, 25, 26, 28 in which instead of a lesion the method is used to detect a pregnancy.
33. The method of paragraph 11 in which the labeled material is introduced into a patient to image a lesion expressing an accumulation of AFPr.
34. The method of paragraph 11 in which the radioactive material is injected into a patient bearing one or more lesion expressing AFPr so that the isotopic decay of the accumulated radioactivity in the lesion will induce cell death.
35. An active binding sequence of mammalian alpha-fetoprotein (AFP) to the receptor of AFP (RECAF), the sequence being Xaa₁ Lys-Gln-Glu-Xaa₂-Leu-Ile-Asn (SEQ. ID. NO: 30), wherein Xaa₁ means any amino acid and Xaa₂ represents Phe or Leu which is the amino acid sequence for the human and mouse AFP fragment respectively, this fragment binding a site of the AFP receptor; this peptide being usable as a substitute for AFP in the detection, purification and imagining of RECAF; this peptide as it binds to RECAF which is elevated in cancer cells allows for a method of diagnostic determination of cancer or chemotherapeutic delivery using cytotoxic or radiological drugs.

1. A peptide comprising the sequence Lys-Glx-Glx-Xaa-Leu-Ile-Asn (SEQ. ID. NO: 1) where Glx means Gln or Glu, each Glx being selected independently of the other, and Xaa represents Phe or Leu, and variants thereof, that bind to the AFP receptor.

2. A peptide according to claim 1, wherein the sequence differs from SEQ. ID. NO: 1 by having at least one insertion, substitution and/or deletion of amino acids in the heptapeptide motif of SEQ. ID, NO: 1, such that at least 4 contiguous amino acids of the heptapeptide motif are maintained in the same order as in SEQ. ID. NO: 1.

3. A peptide according to claim 2, comprising the heptapeptide motif Lys-Glx-Glx-Xaa-Ile-Asp-Leu (SEQ. ID. NO: 2), wherein where Glx means Gln or Glu, each Glx being selected independently of the other, and Xaa represents Phe or Leu.

4-8. (canceled)

9. A peptide according to claim 56, wherein the peptide is conjugated to a molecule of interfering ribonucleic acid (iRNA) or small interfering ribonucleic acid (siRNA), or to an oncolytic virus or to a second peptide or a precursor or vector therefor, wherein the second peptide after delivery to the cell is then expressed on Class I or Class II MHC to elicit CD8+ or CD4+ cell cytotoxicity respectively.

10. A peptide according to claim 56, wherein the peptide is conjugated to a liposome, optionally carrying other agents, for example radioactive isotopes for imaging and therapy, enzymes, toxins, drugs, iRNA, viruses or other agents allow to detect or kill cells expressing AFRr.

11-14. (canceled)

15. A peptide according to claim 10, wherein one or more amino acid is added to the N-terminal end, the C-terminal end or both ends to render the peptide more or less soluble as needed for a particular application, the said one or more amino acids being optionally selected from the twenty naturally occurring amino acids, as well as other amino acids such as homocystine, ornithine, and hydroxyproline; the said addition of the one or more amino acids being optionally achieved through sequential F-MOC synthesis steps or by adding presynthesized groups of amino acids.

16-18. (canceled)

19. A peptide according to claim 15, wherein biotin or other suitable labels are added to enable detecting of the peptide.

20. (canceled)

21. A peptide according to claim 10, wherein the AFPr expressing cells are inside the patient and the materials are capable of being introduced into the patient.

22-23. (canceled)

24. A peptide according to claim 21, adapted to carry toxic or radioactive agents to tumor or inflammatory cells.

25. A polymer or aggregate of the peptides according to claim 24, expressing more than one AFPr binding site, in which all the peptide chains are the same.

26. (canceled)

27. Liposomes comprising a peptide according to claim 1 or a polymer or aggregate for killing tumor cells.

28. Viruses expressing on a capsid thereof a peptide according to claim 1 or a polymer or aggregate for killing tumor cells.

29. iRNA, RNA or DNA conjugated to a peptide according to claim 1 or a polymer or aggregate for delivery to cells to modify cell processes and/or kill tumor cells.

30-33. (canceled)

34. A method for purifying AFPr which comprises binding said AFPr to the material according to claim 1, then separating the peptide/AFPr complex, that results from this binding interaction, from the mixture, and then obtaining AFPr from the complex in relatively pure form.

35. A method for detecting AFPr in which the material according to claim 1 is reacted with material containing AFPr to form a peptide/AFPr complex, and the complex is then detected.

36-47. (canceled)

48. A method according to claim 35, for use in detecting the presence or absence of a lesion which is a benign tumor.

49-50. (canceled)

51. A method according to claim 48, for use with animals, including but not limited to domestic pets, or humans.

52-55. (canceled)

56. A peptide according to claim 4, which consists of or contains any of the following sequences:
(SEQ. ID. NO: 3)
KQEFLIN;
any fragment from the 609 amino acid sequence of
SEQ. ID. NO: 4), provided that the sequence KQEFLIN
(SEQ. ID. NO: 3) is conserved;
(SEQ. ID. NO: 5)
HKDLCQAQGVALQTMKQEFLIN;
(SEQ. ID. NO: 6)
LQTMKQEFLIN;
(SEQ. ID. NO: 7)
TMKQEFLIN;
(SEQ. ID. NO: 8)
LQTMKQELLIN;
(SEQ. ID. NO: 9)
KQELLIN;
(SEQ. ID. NO: 10)
KEEFLIN
(SEQ. ID. NO: 11)
KEQFLIN;
(SEQ. ID. NO: 12)
KQQFLIN;
(SEQ. ID. NO: 13)
KQQFIDL;
(SEQ. ID. NO: 14)
KQQLIDL ;
(SEQ. ID. NO: 29)
KQEFLINLK.

57. A peptide according to claim 56 conjugated directly or indirectly to a surface, a non-peptide molecule, a label, a cytotoxic, radiological or other bioactive agent, or any combination thereof.

58. A method to detect AFPr in which a suitable support membrane is coated with a band of one of the materials described in claim 1 and the sample is mixed with AFPr labeled with a suitable label such as but not limited to colloidal gold, iron particles, iron nanoparticles, colored latex particles and fluorophores and subject to a lateral flow chromatography in the support membrane.

59. A method according to claim 35 for use in detecting the presence or absence of a lesion selected from a malignant tumor or leukemia.


SEQUENCE LISTING (30 sequences)

SEQ ID NO: 1 - protein, 7 aa, Artificial Sequence
  Synthetic peptide
KZZXLIN

SEQ ID NO: 2 - protein, 7 aa, Artificial Sequence
  Synthetic peptide
KZZXIDL

SEQ ID NO: 3 - protein, 7 aa, Artificial Sequence
  Synthetic peptide
KQEFLIN

SEQ ID NO: 4 - protein, 609 aa, Artificial Sequence
  Synthetic peptide
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 5 - protein, 22 aa, Artificial Sequence
  Synthetic peptide
HKDLCQAQGVALQTMKQEFLIN

SEQ ID NO: 6 - protein, 11 aa, Artificial Sequence
  Synthetic peptide
LQTMKQEFLIN

SEQ ID NO: 7 - protein, 9 aa, Artificial Sequence
  Synthetic peptide
TMKQEFLIN

SEQ ID NO: 8 - protein, 11 aa, Artificial Sequence
  Synthetic peptide
LQTMKQELLIN

SEQ ID NO: 9 - protein, 7 aa, Artificial Sequence
  Synthetic peptide
KQELLIN

SEQ ID NO: 10 - protein, 7 aa, Artificial Sequence
  Synthetic peptide
KEEFLIN

SEQ ID NO: 11 - protein, 7 aa, Artificial Sequence
  Synthetic peptide
KEQFLIN

SEQ ID NO: 12 - protein, 7 aa, Artificial Sequence
  Synthetic peptide
KQQFLIN

SEQ ID NO: 13 - protein, 7 aa, Artificial Sequence
  Synthetic peptide
KQQFIDL

SEQ ID NO: 14 - protein, 7 aa, Artificial Sequence
  Synthetic peptide
KQQLIDL

SEQ ID NO: 15 - protein, 9 aa, Artificial Sequence
  Synthetic peptide
CCRDGVLDC

SEQ ID NO: 16 - protein, 9 aa, Artificial Sequence
  Synthetic peptide
EMTXVNXGQ

SEQ ID NO: 17 - protein, 24 aa, Artificial Sequence
  Synthetic peptide
HKDKDLCQAQGVALQTMKQEFLIN

SEQ ID NO: 18 - protein, 19 aa, Artificial Sequence
  Synthetic peptide
VNPGVGQCCTSSYANRRPC

SEQ ID NO: 19 - protein, 22 aa, Artificial Sequence
  Synthetic peptide
FSSLVVDETYVPPAFSDDKFIF

SEQ ID NO: 20 - protein, 11 aa, Artificial Sequence
  Synthetic peptide
HKDLCQAQGVA

SEQ ID NO: 21 - protein, 21 aa, Artificial Sequence
  Synthetic peptide
PAFSDDKFIFHKDLCQAQGVA

SEQ ID NO: 22 - protein, 6 aa, Artificial Sequence
  Synthetic peptide
QTMKQE

SEQ ID NO: 23 - protein, 9 aa, Artificial Sequence
  Synthetic peptide
VALQTMKQE

SEQ ID NO: 24 - protein, 9 aa, Artificial Sequence
  Synthetic peptide
QEFLINLVK

SEQ ID NO: 25 - protein, 5 aa, Artificial Sequence
  Synthetic peptide
KFLIN

SEQ ID NO: 26 - protein, 4 aa, Artificial Sequence
  Synthetic peptide
KFLI

SEQ ID NO: 27 - protein, 4 aa, Artificial Sequence
  Synthetic peptide
KLLI

SEQ ID NO: 28 - protein, 5 aa, Artificial Sequence
  Synthetic peptide
KLLIN

SEQ ID NO: 29 - protein, 9 aa, Artificial Sequence
  Synthetic peptide
KQEFLINLK

SEQ ID NO: 30 - protein, 8 aa, Artificial Sequence
  Synthetic peptide
XKQEXLIN